(19) United States
(12) Patent Application Publication
(10) Pub. No.: US 2018/0094309 A1
(43) Pub. Date: Apr. 5, 2018

(54) NUCLEIC ACID RETRO-ACTIVATED PRIMERS

(71) Applicant: President and Fellows of Harvard College, Cambridge, MA (US)

(72) Inventors: Xi Chen, West Newton, MA (US); Mengmeng Zhang, Newton, MA (US); Peng Yin, Brookline, MA (US)

(73) Assignee: President and Fellows of Harvard College, Cambridge, MA (US)

(21) Appl. No.: 15/564,060

(22) PCT Filed: Apr. 1, 2016
(86) PCT No.: PCT/US16/25480

§ 371 Date: Oct. 3, 2017

Related U.S. Application Data
(60) Provisional application No. 62/142,813, filed on Apr. 3, 2015.
(60) Provisional application No. 62/221,905, filed on Sep. 22, 2015.

(51) Int. Cl. C12Q 1/6853; C12Q 1/686; C12Q 1/6874

(52) U.S. Cl. CPC C12Q 1/6853; C12Q 1/686; C12Q 1/6874; C12Q 2600/16; C12Q 2527/107; C12Q 2535/122; C12Q 2525/161

(57) ABSTRACT

Aspects of the present disclosure are directed to nucleic acid primers, compositions and kits containing the primers, and methods for using the primers in applications requiring, for example, single-molecule sensitivity, single-nucleotide specificity, and/or multiplexed amplification.

### RELATED APPLICATION

This application claims the benefit under 35 U.S.C. § 119(e) of U.S. provisional application No. 62/142,813, filed Apr. 3, 2015, and U.S. provisional application No. 62/221,905, filed Sep. 22, 2015, each of which is incorporated by reference herein in its entirety.

### FEDERALLY SPONSORED RESEARCH

This invention was made with government support under N00014-13-1-0593 awarded by U.S. Department of Defense Office of Naval Research, under OD007292 and EB018659 awarded by National Institutes of Health, and under CCF-1054898 and CCF-1317291 awarded by National Science Foundation. The government has certain rights in the invention.

### BACKGROUND OF INVENTION

Primers are widely used in various nucleic acid amplification reactions, including polymerase chain reaction (PCR). Nonetheless, the traditional design of a primer (e.g., a short single-stranded DNA with an extendable 3′ end) has drawbacks; for example, it is prone to creating unwanted amplification products (also known as PCR artifacts) such as non-specific amplicons and primer dimers.

### SUMMARY OF INVENTION

Provided herein are nucleic acid primers, referred to as “retro-activated primers” (“RA primers”), for use in applications requiring, for example, single-molecule sensitivity, single-nucleotide specificity, and/or multiplexed amplification. RA primers of the present disclosure contain a “sensing module” and a “priming module” (FIG. 1A). The sensing module functions to determine whether a nucleic acid is a target of interest. The priming module functions to prime a target nucleic acid for synthesis. When the sensing module binds to a target nucleic acid, it recruits and activates the priming module to bind to the target nucleic acid (i.e., “prime”), upstream of the sensing module (FIG. 1B). Advantageously, in the absence of recruitment and activation by the sensing module, the priming module remains inactive, preventing or minimizing “mis-priming” (e.g., binding to a nucleic acid that is not a target nucleic acid) and the creation of, for example, PCR artifacts.

Also provided herein is a two-step method that uses RA primers and dsBlocker primers (International Pub. No. WO 2015/010020, incorporated herein by reference) for selective amplification of DNA. The two-step method, in some embodiments, includes an “adaptor tagging” step and a “mutation enrichment” step. In the adaptor tagging step, multiple (e.g., 10, 20, 30, 40, 50, 60, 70, 80, 90 or 100) different genome loci (e.g., each 30-50 base pair (bp) long) undergo various cycles of PCR in the presence a large set of primers that append a single-molecule barcode, sample index and sequencing primers (collectively referred to as “adaptors”) to the target sequence. In the mutation enrichment stage, a dsBlocker primer and a RA primer may be used in combination to selectively amplify target DNA that, for example, contains a mutation (e.g., relative to a wild-type target DNA) (FIG. 8C).

RA primers of the present disclosure may be used in a variety of nucleic acid detection and synthesis reactions. For example, the present disclosure contemplates the use of RA primers for detecting rare circulating tumor DNA (ctDNA). Detection and analysis of solid tumor from a simple blood draw (a concept referred to as ‘liquid biopsy’) is a desired capability in cancer care. When achieved with sufficient accuracy, it may address several unmet medical needs, such as confirming suspected cancer cases from CT/MRI-based screening, monitoring the effectiveness of treatments in real time, and guiding therapeutic actions in cancers that fail the initial treatment, for example. While several circulating biomarkers, such as circulating tumor cells (CTCs) and circulating microRNA, have gained considerable attention, circulating tumor DNA (ctDNA) has been validated most thoroughly. ctDNA can be distinguished from cell-free DNA (cfDNA) obtained from healthy cells due to the presence of cancer-specific genetic alterations, such as sequences, mutations and rearrangements. Using ultrasensitive methods, such as, for example, digital PCR and NextGen Sequencing (NGS), ctDNA has been shown to be extremely valuable in the applications mentioned above.

While several technology platforms are successful in research settings, their implementation within clinical practice is hindered by their respective drawbacks. In particular, probe-based methods, such as allele-specific PCR (AS-PCR), digital PCR, and BEAMing, require a dedicated probe (e.g., a TaqMan probe) to detect a particular mutation.

Therefore, such methods can only detect ctDNA from patients having tumors that carry highly recurrent oncogene mutations, which represent only a small fraction of patients in many types of cancer. For instance, only 3% of lung squamous cell carcinomas carry these mutations. On the other hand, NGS-based methods often have high raw error rates (often 0.1% to 1%) and complex workflow. For example, one standard-setting protocol (Newman et al. Nature Medicine 20, 548-554 (2014)) involves approximately 40 liquid transfer steps and takes more than a week to complete. Moreover, the cost of NGS-based ctDNA testing is prohibitive for repeat testing. A typical cancer panel sequenced at 100,000× depth (as necessitated by the rarity of ctDNA and high raw error rate) would require approximately 10 M reads.

The present disclosure provides RA primers, methods, compositions and kits to, for example, reduce the cost and complexity associated with NGS-based ctDNA testing (as well as other target testing). This is achieved, in some embodiments, by enriching the fraction of mutant DNA to sufficient abundance (e.g., >10%) so that the relatively high raw error rate is tolerable and fewer reads are generated, and by completing the sample preparation without complicated hybridization and/or bead-based separation steps. Further, in some embodiments, the RA primers of the present disclosure permit the parallel, multiplexed enrichment of thousands of possible mutations in approximately 100 genomic loci.

Thus, provided herein, in some embodiments, are RA primers and methods that permit target selection to be completed in a simple, PCR-like reaction. Such methods are inherently robust to temperature variation and interfering DNA, and thus can be multiplexed and/or parallelized for high-throughput sample preparation.

Some aspects of the present disclosure provide primers that comprise a sensing module and a priming module, wherein the sensing module binds to a target nucleic acid and, when bound to the target nucleic acid, recruits and activates the priming module, wherein the activated priming module binds to the target nucleic acid upstream of the sensing module.

In some embodiments, the sensing module comprises a partially double-stranded nucleic acid comprising a first nucleic acid strand bound to a second nucleic acid strand, wherein the first strand comprises (a) a 5′ domain that includes sequence complementary to the priming module, wherein a portion of the sequence of (a) is complementary to and bound to the second strand, thereby forming a double-stranded region, and (b) a 3′ domain that includes sequence complementary to the target nucleic acid, wherein a portion of the sequence of (b) is complementary to and bound to the second strand, thereby forming a double-stranded region.

In some embodiments, the priming module comprises a partially double-stranded nucleic acid comprising a first nucleic acid strand bound to a second nucleic acid strand, wherein the first strand comprises (c) a 5′ domain that includes sequence complementary to the sensing module, wherein a portion of the sequence of (c) is complementary to and bound to the second strand, thereby forming a double-stranded region, and (d) a 3′ domain that includes, or optionally includes, a chemical linker attached to a minimal primer sequence, wherein the minimal primer sequence is complementary to the target nucleic acid and is bound to the second strand, thereby forming a double-stranded region.

In some embodiments, the priming module comprises a partially double-stranded nucleic acid comprising a first nucleic acid strand bound to a second nucleic acid strand, wherein the first strand comprises (c) a 5′ domain that includes sequence complementary to the sensing module, wherein a portion of the sequence of (c) is complementary to and bound to the second strand, thereby forming a double-stranded region, and (d) a 3′ domain attached to a minimal primer sequence, wherein the minimal primer sequence is complementary to the target nucleic acid and is bound to the second strand, thereby forming a double-stranded region.

In some embodiments, the sensing module comprises (a) a first nucleic acid strand containing, in a 5′ to 3′ direction, Domain 1A, Domain 2A, Domain 3A and Domain 4A, wherein Domain 1A and Domain 4A are unbound, and wherein Domain 3A and Domain 4A are complementary to the target nucleic acid, and (b) a second nucleic acid strand containing, in a 5′ to 3′ direction, Domain 3B and Domain 2B, wherein Domain 3B and Domain 2B are respectively complementary to and bound to Domain 3A and Domain 2A of the first strand of (a).

In some embodiments, the priming module comprises (c) a first nucleic acid strand containing, in a 5′ to 3′ direction, Domain 2B, Domain 1B, Domain 5A, optionally a linker molecule, and Domain 6A, wherein Domain 2B and Domain 1B are respectively complementary to Domain 2A and Domain 1A of the first strand of (a), and Domain 2B is unbound, and (d) a second nucleic acid containing, in a 5′ to 3′ direction, Domain 6B, Domain 7, Domain 5B and Domain 1A, wherein Domain 6B, Domain 5B and Domain 1A are respectively complementary to and bound to Domain 6A, Domain 5A and Domain 1B of the first strand of (c), and wherein Domain 7 is optionally unbound.

In some embodiments, Domain 5A and Domain 6A are separated from each other by a polymerase-stopping or a polymerase-pausing moiety.

In some embodiments, the sensing module is linked to the priming module via a linker molecule. In some embodiments, the linker molecule is a chemical linker. In some embodiments, the linker molecule is a single-stranded nucleic acid.

Some aspects of the present disclosure provide a nucleic acid molecule, comprising (a) a first nucleic acid strand, (b) a second nucleic acid strand comprising (i) a 3′ domain that includes sequence complementary to and bound to the first strand, thereby forming a first double-stranded domain, and (ii) a 5′ domain that includes sequence complementary to and bound to a third nucleic acid strand, thereby forming a second double-stranded domain, and (c) the third nucleic acid strand comprising (i) a 5′ domain that contributes to the second double-stranded domain of (b)(ii), and (ii) a 3′ domain that includes (or optionally includes) a chemical linker attached to a minimal primer sequence, wherein the minimal primer sequence is complementary to the target nucleic acid.

In some embodiments, a nucleic acid molecule comprises (a) a first nucleic acid strand, (b) a second nucleic acid strand comprising (i) a 3′ domain that includes sequence complementary to and bound to the first strand, thereby forming a first double-stranded domain, and (ii) a 5′ domain that includes sequence complementary to and bound to a third nucleic acid strand, thereby forming a second double-stranded domain, and (c) the third nucleic acid strand comprising (i) a 5′ domain that contributes to the second double-stranded domain of (b)(ii), and (ii) a 3′ domain attached to a minimal primer sequence, wherein the minimal primer sequence is complementary to the target nucleic acid.

In some embodiments, the minimal primer sequence is bound to the first strand, upstream from the first double-stranded region.

Also provided herein are methods that comprise combining in a reaction mixture a target nucleic acid and any of the primers, as provided herein.

Some aspects of the present disclosure provide methods that comprise combining in a reaction mixture a target nucleic acid and a primer that comprises a sensing module and a priming module, wherein the sensing module binds to a target nucleic acid and, when bound to the target nucleic acid, recruits and activates the priming module, wherein the activated priming module binds to the target nucleic acid upstream of the sensing module.

In some embodiments, the methods comprise combining in a reaction mixture a target nucleic acid with a primer that comprises a sensing module and a priming module, wherein the sensing module comprises a partially double-stranded nucleic acid comprising a first nucleic acid strand bound to a second nucleic acid strand, wherein the first strand comprises (a) a 5′ domain that includes sequence complementary to the priming module, wherein a portion of the sequence of (a) is complementary to and bound to the second strand, and (b) a 3′ domain that includes sequence complementary to the target nucleic acid, wherein a portion of the sequence of (b) is complementary to and bound to the second strand, and the priming module comprises a partially double-stranded nucleic acid comprising a first nucleic acid strand bound to a second nucleic acid strand, wherein the first strand comprises (c) a 5′ domain that includes sequence complementary to the sensing module, wherein a portion of the sequence of (c) is complementary to and bound to the second strand, and (d) a 3′ domain that includes (or optionally includes) a chemical linker attached to a minimal primer sequence, wherein the minimal primer sequence is complementary to the target nucleic acid and is bound to the second strand.

In some embodiments, the methods further comprise incubating the reaction mixture under conditions that result in recruitment of the primer module to the sensing module, activation of the priming module, and binding of the minimal primer sequence to the target nucleic acid.

In some embodiments, the methods further comprise incubating the reaction mixture under conditions that result in amplification of the target nucleic acid.

Also provided herein are compositions comprising any of the primers or nucleic acid molecules, as provided herein.

In some embodiments, the compositions further comprise target nucleic acid.

Also provided herein are kits comprising any of the primers or nucleic acid molecules, as provided herein.

In some embodiments, the kits comprise at least two of the primers (e.g., RA primers) as provided herein, wherein each primer is designed to bind to a different target nucleic acid.

In some embodiments, the kits further comprise at least one of the following reagents: buffer, deoxyribonucleotide triphosphates (dNTPs), nuclease-free water and polymerase.

### BRIEF DESCRIPTION OF DRAWINGS

The accompanying drawings are not intended to be drawn to scale. For purposes of clarity, not every component may be labeled in every drawing. For all schematics depicted in the figures, a dot represents the 5′ end of a nucleic acid, and a triangle represents the 3′ end of the same nucleic acid. An inward triangle represents the presence of an optional modification at the 3′ end of a nucleic acid that prevents extension.

FIGS. 1A and 1B depict an example of a retro-activated (RA) primer of the present disclosure.

FIG. 2 depicts an example of a process of unfavorable artifact generation using RA primers of the present disclosure.

FIG. 3 depicts the a simulation of the amplification kinetics of a nucleic acid template having a starting concentration of 1 pM. The forward primer is an RA primer of the present disclosure. The probability of the bottom strand of the non-target nucleic acid being copied by the forward primer varies from 1 to 10⁻⁵. The probability of the top strand of the non-target nucleic acid being copied by the reverse primer is set to be invariably 1.

FIGS. 4A and 4B depict various nucleic acid domains of the priming module and the sensing module. The “pro-anchor” strand contains sequence (Domains “3A”+“4”) that is complementary to and binds to a target nucleic acid of interest and sequence (Domains “1A”+“2A”) that is complementary to and binds to the “pro-primer” strand of the priming module. The pro-primer strand, likewise, contains sequence (Domains “2B*”+“1B*”) that is complementary to and binds to the pro-anchor strand. The pro-primer strand also contains sequence (Domain “6A”) that is complementary to and binds to the target nucleic acid of interest. FIGS. 4C-4F depict several examples of retro-activated (RA) primers. The thick gray arrows represent the minimal primer. The strand ‘1’ in FIG. 4C and corresponding strands in other panels represent the template. The molecular complex ‘2’ in FIG. 4C and corresponding molecules or molecular complexes in other panels represent the sensing module. The molecular complex ‘3’ in FIG. 4C and corresponding molecules or molecular complexes in other panels represent the priming module.

FIG. 5 depicts a mechanism of action of an RA primer of the present disclosure.

FIGS. 6A-6C depict the amplification kinetics of a target of interest (solid lines) and a non-target nucleic acid (dashed lines) from quantitative polymerase chain reaction (PCR) reactions using different primer designs. The primers used in the experiments represented by the graphs in FIGS. 6A and 6B are designed based on those described in International Pub. No. WO/2015/010020. The primers used in the experiments represented by the graph in FIG. 6C are RA primers of the present disclosure—the forward and reverse minimal primers are both 13 nucleotides in length.

FIGS. 7A and 7B show selective amplification of mutant DNA using an RA primer of the present disclosure used in combination with a primer based on the dsBlocker as described in International Pub. No. WO/2015/010020 (also referred to herein as an “iClamp”). FIG. 7A: without dsBlocker; FIG. 7B: with dsBlocker. In both panels: solid line and dashed line show the amplification kinetics of (template A) and (template A2), respectively.

FIGS. 8A-8C depict various components of an iWISE library-construction platform of the present disclosure.

FIG. 9 shows data from a qPCR-based assessment of iWISE-PCR efficiency.

FIG. 10 shows examples of dsBlockers of the present disclosure.

FIG. 11 shows PCR amplification using a dsBlocker of the present disclosure.

FIG. 12 shows that when the annealing temperature is changed from 68° C. to 53° C. (a 15° C. difference), RA primers can be used to efficiently amplify the intended targets. Solids lines represent the reactions with templates, and the dotted lines represent the no template controls.

FIG. 13 shows that RA primers can detect the template with single copy number in the reactions. Changing the temperatures does not influence this single molecule sensitivity. Shown here, the annealing temperature is changed from 58° C. to 68° C. (a 10° C. difference), yet the single molecule sensitivity remains across all temperatures selected between the two temperatures. Different shades of gray represent different copy numbers of the template. The template with single copy number was tested in 6 reactions due to Poisson distribution.

### DETAILED DESCRIPTION OF INVENTION

Aspects of the present disclosure are directed to nucleic acid primers, referred to as “retro-activated primers,” or “RA primers,” methods using the RA primers, and compositions and kits containing the RA primers. RA primers are designed to address the problem of “mis-priming” (e.g., primer binding to a non-target nucleic acid). Also provided herein are primers, methods, compositions and kits for selective amplification of DNA (e.g., mutant DNA) using RA primers in combination with dsBlocker primers (as described, for example, in International Pub. No. WO/2015/010020, incorporated herein by reference).

### Retro-Activated Primers (RA Primers)

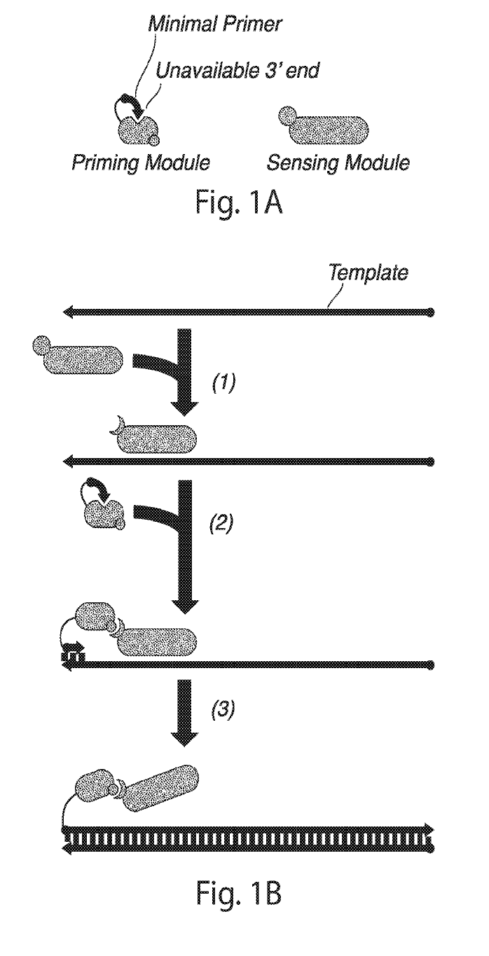

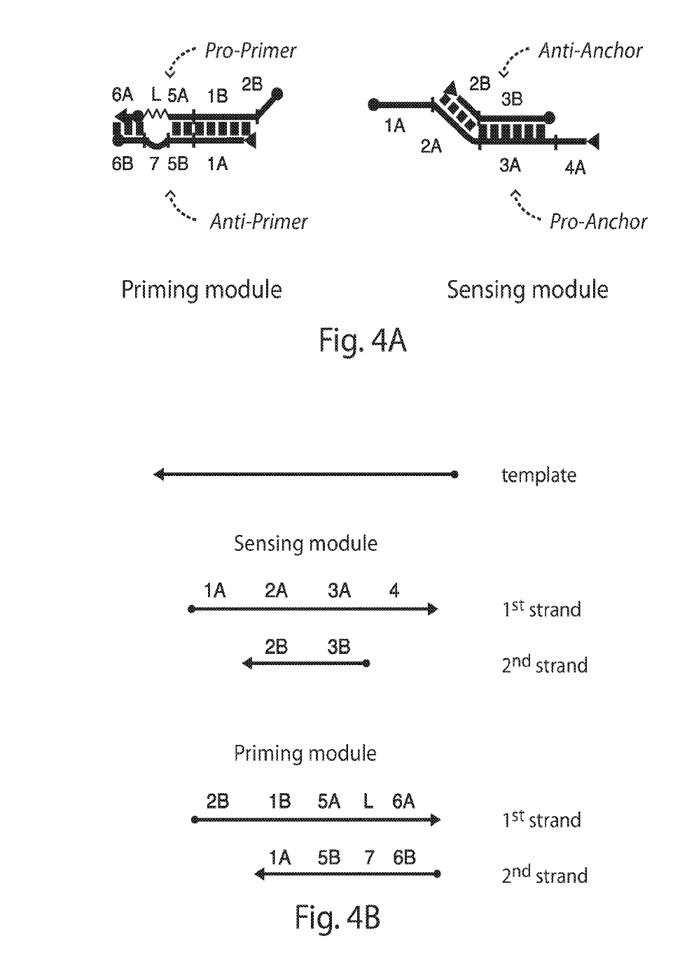

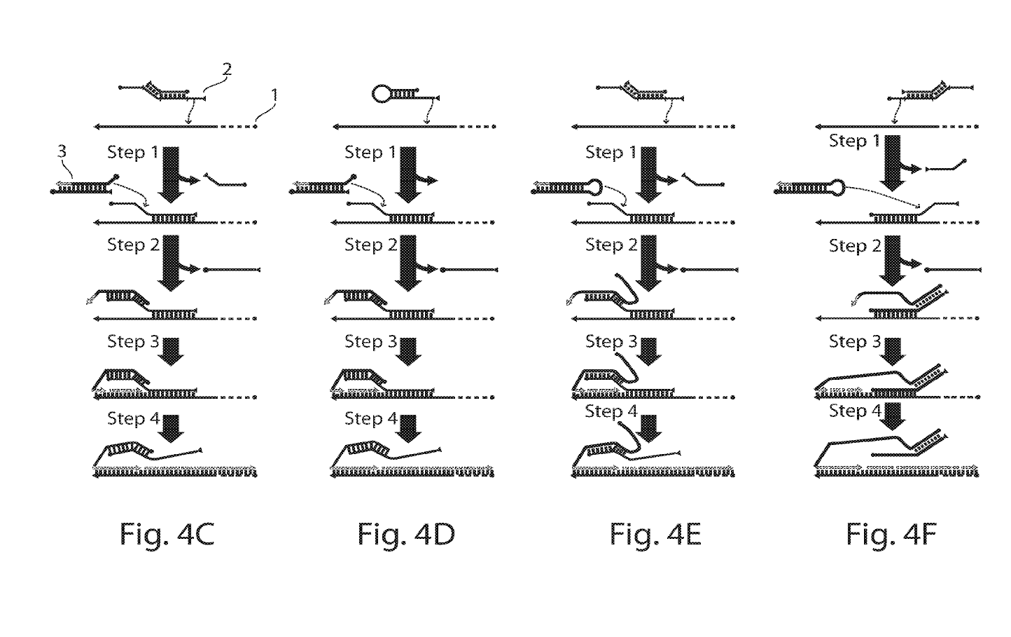

Examples of RA primers of the present disclosure are depicted in FIGS. 1A, 1B and FIGS. 4A-4F. As shown in FIG. 1A, an RA primer includes a “priming module” (left) and a “sensing module” (right). When a sensing module binds to a target nucleic acid of interest (FIG. 1B(1)), it recruits and activates the priming module (FIG. 1B(2)), which then binds to the target via a 3′ minimal primer domain (FIG. 1A, left, and FIG. 1B(3)), and the target may be copied, for example. As the target is copied and the primer is extended, the newly formed extension displaces the sensing module (FIG. 1B(3). In its soluble form (that is, when it is not bound to the sensing module), in most instances, the 3′ minimal primer domain of the priming module is sequestered, and the priming module remains inactive and unable to bind to another nucleic acid (FIG. 1A and FIG. 4 (left panel, Domain “6A”)).

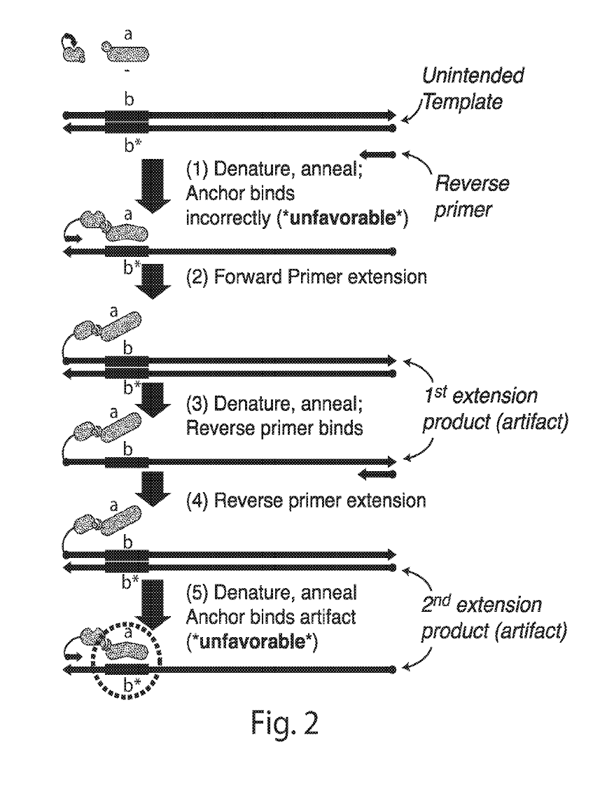

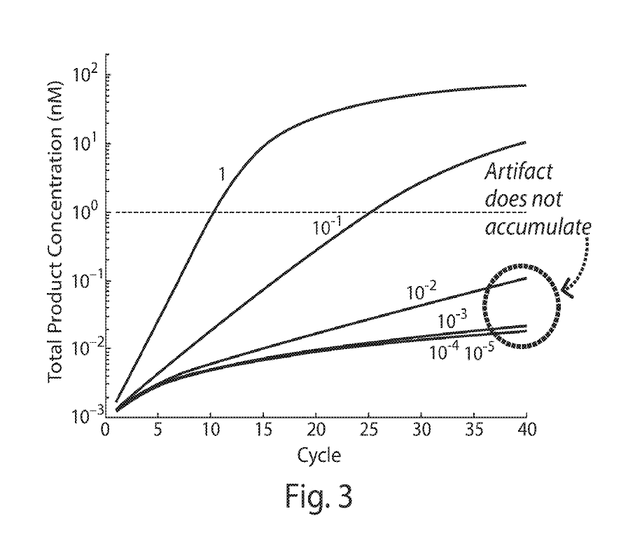

In some cases, the sensing module may inadvertently bind a non-target nucleic acid, and recruit and activate the priming module to copy the non-target nucleic acid (FIGS. 2(1) and 2(2)). That is, an RA primer may still “mis-prime.” Even so, when the nucleic acid product produced as result of this mis-priming event is copied in the next amplification cycle (FIGS. 2(3) and 2(4)), for example, the newly synthesized strand contains the sequence of the non-target nucleic acid, rather than the target nucleic acid. Thus, in subsequent cycles, amplification of the mis-primed product remains unfavorable (FIG. 2(5)). Reducing the mis-priming probability to a reasonable level (e.g., 10⁻²) effectively eliminates the accumulation of non-specific amplification products (FIG. 3).

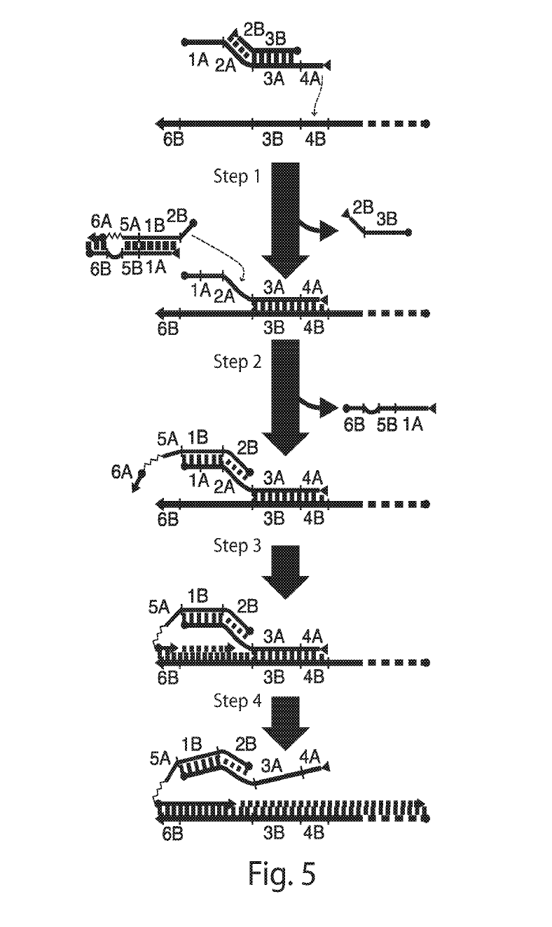

FIG. 4A depicts various domains of an example a priming module and the sensing module of an RA primer of the present disclosure. In FIGS. 4A and 4B, the sensing module comprises two nucleic acid strands: a first strand, referred to as the “pro-anchor strand,” and a second strand, referred to as the “anti-anchor strand” strand. In some embodiments, the second strand of the sensing module is shorter than (e.g., at least 5, 10, 15, 20 or 25 nucleotides shorter than) the first strand. In FIGS. 4A and 4B, the priming module also comprises two nucleic acid strands: a first strand, referred to as the “pro-primer strand,” and a second strand, referred to as the “anti-primer strand.” In some embodiments, the second strand of the priming module is shorter than (e.g., at least 5, 10, 15, 20 or 25 nucleotides shorter than) the first strand. In FIG. 4A, the triangle at the end of pro-primer indicates a chemically extendable 3′ end; the inverted triangle at the end of other strands indicate a blocked (i.e. non-extendable) 3′ end to prevent unwanted extension of these strands. The mechanism of action of this example RA primer is depicted in FIG. 5.

The lengths of the pro-anchor strand and the anti-anchor strand may vary.

In some embodiments, a pro-anchor strand (e.g., the first strand of the sensing module) has length of 20 to 100 nucleotides. For example, a pro-anchor strand may have a length of 20 to 90, 20 to 80, 20 to 70, 20 to 60, 20 to 50, 20 to 40, or 20 to 30 nucleotides. In some embodiments, a pro-anchor strand has a length of 30 to 90, 30 to 80, 30 to 70, 30 to 60, 30 to 50, 30 to 40, 40 to 90, 40 to 80, 40 to 70, 40 to 60, 40 to 50, 50 to 90, 50 to 80, 50 to 70, or 50 to 60 nucleotides. In some embodiments, a pro-anchor strand has a length of 40, 41, 42, 43, 44, 45, 46, 47, 48, 49, 50, 51, 52, 53, 54, 55, 56, 57, 58, 59 or 60 nucleotides. In some embodiments, the pro-anchor strand may be shorter than 20 nucleotides, while in other embodiments, it is longer than 100 nucleotides.

In some embodiments, an anti-anchor strand (e.g., the second strand of the sensing module) has length of 10 to 100 nucleotides. For example, an anti-anchor strand may have a length of 10 to 90, 10 to 80, 10 to 70, 10 to 60, 10 to 50, 10 to 40, 10 to 30, or 10 to 20 nucleotides. In some embodiments, an anti-anchor strand has a length of 20 to 90, 20 to 80, 20 to 70, 20 to 60, 20 to 50, 20 to 40, 20 to 30, 30 to 90, 30 to 80, 30 to 70, 30 to 60, 30 to 50, 30 to 40, 40 to 90, 40 to 80, 40 to 70, 40 to 60, 40 to 50, 50 to 90, 50 to 80, 50 to 70, or 50 to 60 nucleotides. In some embodiments, an anti-anchor strand has a length of 40, 41, 42, 43, 44, 45, 46, 47, 48, 49, 50, 51, 52, 53, 54, 55, 56, 57, 58, 59 or 60 nucleotides. In some embodiments, the anti-anchor strand may be shorter than 10 nucleotides, while on other embodiments, it is longer than 100 nucleotides.

In some embodiments, an anti-anchor strand (e.g., the second strand) is shorter than a pro-anchor strand (e.g., the first strand) of the sensing module. For example, an anti-anchor strand may be 5% to 80% shorter than a pro-anchor strand. In some embodiments, an anti-anchor strand is 10% to 80%, 20% to 70%, 30% to 60%, or 40% to 50% shorter than a pro-anchor strand. In some embodiments, an anti-anchor strand is 10%, 15%, 20%, 25%, 30%, 35%, 40%, 45%, 50%, 55%, 60%, 65%, 70%, 75%, or 80% shorter than a pro-anchor strand.

The lengths of the pro-primer strand and the anti-primer strand may vary.

In some embodiments, a pro-primer strand (e.g., the first strand of the priming module) has length of 20 to 100 nucleotides. For example, a pro-primer strand may have a length of 20 to 90, 20 to 80, 20 to 70, 20 to 60, 20 to 50, 20 to 40, or 20 to 30 nucleotides. In some embodiments, a pro-primer strand has a length of 30 to 90, 30 to 80, 30 to 70, 30 to 60, 30 to 50, 30 to 40, 40 to 90, 40 to 80, 40 to 70, 40 to 60, 40 to 50, 50 to 90, 50 to 80, 50 to 70, or 50 to 60 nucleotides. In some embodiments, a pro-primer strand has a length of 40, 41, 42, 43, 44, 45, 46, 47, 48, 49, 50, 51, 52, 53, 54, 55, 56, 57, 58, 59 or 60 nucleotides. In some embodiments, the pro-primer strand may be shorter than 20 nucleotides, while in other embodiments, it is longer than 100 nucleotides.

In some embodiments, an anti-primer strand (e.g., the second strand of the priming module) has length of 10 to 100 nucleotides. For example, an anti-primer strand may have a length of 10 to 90, 10 to 80, 10 to 70, 10 to 60, 10 to 50, 10 to 40, 10 to 30, or 10 to 20 nucleotides. In some embodiments, an anti-primer strand has a length of 20 to 90, 20 to 80, 20 to 70, 20 to 60, 20 to 50, 20 to 40, 20 to 30, 30 to 90, 30 to 80, 30 to 70, 30 to 60, 30 to 50, 30 to 40, 40 to 90, 40 to 80, 40 to 70, 40 to 60, 40 to 50, 50 to 90, 50 to 80, 50 to 70, or 50 to 60 nucleotides. In some embodiments, an anti-primer strand has a length of 40, 41, 42, 43, 44, 45, 46, 47, 48, 49, 50, 51, 52, 53, 54, 55, 56, 57, 58, 59 or 60 nucleotides. In some embodiments, the anti-primer strand may be shorter than 10 nucleotides, while on other embodiments, it is longer than 100 nucleotides.

In some embodiments, an anti-primer strand (e.g., the second strand) is shorter than a pro-primer strand (e.g., the first strand) of the priming module. For example, an anti-primer strand may be 5% to 80% shorter than a pro-primer strand. In some embodiments, an anti-primer strand is 10% to 80%, 20% to 70%, 30% to 60%, or 40% to 50% shorter than a pro-primer strand. In some embodiments, an anti-primer strand is 10%, 15%, 20%, 25%, 30%, 35%, 40%, 45%, 50%, 55%, 60%, 65%, 70%, 75%, or 80% shorter than a pro-primer strand.

A “minimal primer domain” refers to the domain of the pro-primer strand of the priming module that is capable of binding to a target nucleic acid. In some embodiments, a minimal primer domain as a length of 5 to 25 nucleotides. For example, a minimal primer domain may have a length of 5 to 20, 5 to 15, 5 to 10, 10 to 25, 10 to 20, or 10 to 15 nucleotides. In some embodiments, a minimal primer domain as a length of 5, 6, 7, 8, 9, 10, 11, 12, 13, 14, 15, 16, 17, 18, 19, 20, 21, 22, 23, 24 or 25 nucleotides.

The components of the priming module and the sensing module of an RA primer are described as domains. A nucleic acid “domain” (used interchangeably with the term “region”) refers to a contiguous nucleotide sequence having defined (e.g., rationally-defined) properties (e.g., length, nucleotide composition, complementary relative to another domain on the same or separate molecule, and binding capability). For example, properties of a particular domain may be defined based on the target nucleic acid sequence. In some embodiments, at least some of the domains of an RA primer are designed to avoid unwanted secondary structures (e.g., hairpin loops), to avoid long nucleotide homopolymers (e.g., having a contiguous stretch of 3, 4, 5 or more of the same (e.g., all adenine (A) or all thymine (T)) nucleotides), and/or to avoid high (e.g., greater than 80%) or low (e.g., less than 20%) guanine (G)/cytosine (C) content.

For simplicity, each nucleic acid domain of an RA primer may be described using a number/letter convention, as shown, for example, in FIGS. 4A and 4B.

“Domain 1A” of the pro-anchor strand of the sensing module refers to a domain that is complementary to and binds to the pro-primer strand of the priming module. Domain 1A of the pro-anchor strand is not complementary to and does not bind to the target nucleic acid or the anti-anchor strand. Thus, in the absence of a target nucleic acid, Domain 1A of the pro-anchor strand is unbound, and in the presence of a target nucleic acid, Domain 1A of the pro-anchor strand binds to Domain 1B of the pro-primer strand. A nucleic acid domain is considered “unbound” if it is a single-stranded domain (that is, the domain is not bound to another nucleic acid). It should be understood that while in the absence of a target nucleic acid “unbound” Domain 1A is single-stranded, in the presence of a target nucleic acid, Domain 1A binds to the pro-primer strand, thereby forming a double-stranded domain and is no longer considered “unbound.” “Domain 2A” of the pro-anchor strand of the sensing module refers to a domain that is complementary to and binds to the pro-primer strand of the priming module. Domain 2A of the pro-anchor strand is also complementary to and binds to the anti-anchor strand of the sensing module. Domain 2A of the pro-anchor strand is not complementary to and does not bind to the target nucleic acid. Thus, in the absence of a target nucleic acid, Domain 2A of the pro-anchor strand binds to Domain 2B of the anti-anchor strand, and in the presence of a target nucleic acid, Domain 2A of the pro-anchor strand dissociates from Domain 2B of the anti-anchor strand and binds to Domain 2B of the pro-primer strand of the priming module.

“Domain 3A” of the pro-anchor strand of the sensing module refers to a domain that is complementary to and binds to the target nucleic acid. Domain 3A of the pro-anchor strand is also complementary to and binds to the anti-anchor strand of a sensing module. Domain 3A of the pro-anchor strand is not complementary to and does not bind to the priming module. Thus, in the absence of a target nucleic acid, Domain 3A of the pro-anchor strand binds to Domain 3B of the anti-anchor strand, and in the presence of a target nucleic acid, Domain 3A of the pro-anchor strand dissociates from Domain 3B of the anti-anchors strand and binds to the target nucleic acid.

“Domain 4A” of the pro-anchor strand of the sensing module refers to a domain that is complementary to and binds to the target nucleic acid. Domain 4A of the pro-anchor strand is not complementary to and does not bind to the priming module or the anti-anchor strand of the sensing module. Thus, in the absence of a target nucleic acid, Domain 4A of the pro-anchor strand is unbound, and in the presence of a target nucleic acid, Domain 4A of the pro-anchor strand binds to the target nucleic acid.

“Domain 3B” of the anti-anchor strand of the sensing module refers to a domain that is complementary to and binds to Domain 3A of the pro-anchor strand of the sensing module. Domain 3B of the anti-anchor strand is not complementary to and does not bind to the target nucleic acid or the priming module. Thus, in the absence of a target nucleic acid, Domain 3B of the anti-anchor strand is bound to Domain 3A of the pro-anchor strand, and in the presence of a target nucleic acid, Domain 3B of the anti-anchor strand dissociates from Domain 3A of the pro-anchor strand.

“Domain 2B” of the anti-anchor strand of the sensing module refers to a domain that is complementary to and binds to Domain 2A of the pro-anchor strand of the sensing module. Domain 2B of the anti-anchor strand is not complementary to and does not bind to the target nucleic acid or the priming module. Thus, in the absence of a target nucleic acid, Domain 2B of the anti-anchor strand binds to Domain 2A of the pro-anchor strand, and in the presence of a target nucleic acid, Domain 2B of the anti-anchor strand dissociates from Domain 2A of the pro-anchor strand.

“Domain 2B” of the pro-primer strand of the priming module refers to a domain that is complementary to and binds to the pro-anchor strand of the sensing module. Domain 2B of the pro-primer strand is not complementary to and does not bind to the target nucleic acid or to the anti-primer strand of the priming module. Thus, in the absence of a target nucleic acid, Domain 2B of the pro-primer strand is unbound, and in the presence of a target nucleic acid, Domain 2B of the pro-primer strand binds to Domain 2A of the pro-anchor strand of the sensing module.

“Domain 1B” of the pro-primer strand of the priming module refers to a domain that is complementary to and binds to the pro-anchor strand of the sensing module. Domain 1B of the pro-primer strand is also complementary to and binds to the anti-primer strand but not to the target nucleic acid. Thus, in the absence of a target nucleic acid, Domain 1B of the pro-primer strand binds to Domain 1A of the anti-anchor strand, and in the presence of a target nucleic acid, Domain 1B of the pro-primer strand dissociates from Domain 1A of the anti-primer strand and binds to Domain 1A of the pro-anchor strand of the sensing module.

“Domain 5A” of the pro-primer strand of the priming module refers to a domain that is complementary to and binds to the anti-primer strand of the priming module but not to the sensing module or the target nucleic acid. Thus, in the absence of a target nucleic acid, Domain 5A of the pro-primer strand binds to Domain 5B of the anti-primer strand, and in the presence of a target nucleic acid, Domain 5A of the pro-primer strand dissociates from Domain 5B of the anti-primer strand.

“Domain 6A” of the pro-primer strand of the priming module refers to a domain that is complementary to and binds to the target nucleic acid. Domain 6A is also complementary to and binds to the anti-primer strand of the priming module but not to the sensing module. Thus, in the absence of a target nucleic acid, Domain 6A of the pro-primer strand binds to Domain 6B of the anti-primer strand, and in the presence of a target nucleic acid, Domain 6A of the pro-primer strand dissociates from Domain 6B of the anti-primer strand and binds to the target nucleic acid, upstream of the pro-anchor Domains 3A and 4A. Domain 6A is also referred to as the “minimal primer domain” (FIG. 1A). In some embodiments, the minimal primer domain is linked to the other domains of the pro-primer strand via a linker molecule (e.g., a chemical linker molecule, such as, for example, hexaethylene glycol, polyethylene glycol, an alkyl spacer, a peptide nucleic acid or a linked nucleic acid). Without recruitment of the priming module to the sensing module (e.g., in the absence of a target nucleic acid), the minimal primer domain remains bound to the anti-primer strand and is unable to bind to another nucleic acid.

“Domain 6B” of the anti-primer strand of the priming module refers to a domain that is complementary to and binds to the pro-primer strand of the priming module. Domain 6B is not complementary to and does not bind to the sensing module or the target nucleic acid. Thus, in the absence of a target nucleic acid, Domain 6B of the anti-primer strand of the priming module binds to Domain 6A of the pro-primer strand, and in the presence of a target nucleic acid, Domain 6B of the anti-primer strand dissociates from Domain 6A of the pro-primer strand.

“Domain 7” of the anti-primer strand of the priming module refers to a domain that links Domain 6B to Domain 5B. Domain 7 is not complementary to and does not bind to the pro-primer strand of the priming module, the sensing module, or the target nucleic acid.

“Domain 5B” of the anti-primer strand of the priming module refers to a domain that is complementary to and binds to the pro-primer strand of the priming module. Domain 5B is not complementary to and does not bind to the sensing module or the target nucleic acid. Thus, in the absence of a target nucleic acid, Domain 5B of the anti-primer strand binds to Domain 5A of the pro-primer strand, and in the presence of a target nucleic acid, Domain 5B of the anti-primer strand dissociates from Domain 5A of the pro-primer strand.

“Domain 1A” of the anti-primer strand of the priming module refers to a domain that is complementary to and binds to the pro-primer strand of the priming module. Domain 1A is not complementary to and does not bind to the sensing module or the target nucleic acid. Thus, in the absence of a target nucleic acid, Domain 1A of the anti-primer strand binds to Domain 1B of the pro-primer strand, and in the presence of a target nucleic acid, Domain 1A of the anti-primer strand dissociates from Domain 1B of the pro-primer strand.

In some embodiments, a nucleic acid domain binds transiently to a complementary nucleic acid domain. A nucleic acid domain is considered to bind “transiently” to a complementary nucleic acid domain if it binds to the complementary nucleic acid and then unbinds (dissociates) within a short period of time at a given temperature. By contrast, a nucleic acid domain is considered to bind “stably” to a complementary nucleic acid domain if it binds to the complementary nucleic acid and remains bound to the complementary nucleic acid domain at a given temperature for the length of time of a given reaction (e.g., amplification reaction).

In some embodiments, a nucleic acid domain binds transiently to a complementary nucleic acid domain at room temperature. In some embodiments, a nucleic acid domains binds transiently to a complementary nucleic acid domain at annealing temperature. “Annealing temperature” includes temperatures in the range of 20° C. to 72° C., or 40° C. to 72° C. For example, an annealing temperature may be 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C. or 72° C. In some embodiments, a nucleic acid domain bound transiently to a complementary nucleic acid domain binds to the complementary nucleic acid domain for 0.1 to 10, or 0.1 to 5 seconds. For example, a nucleic acid domain may bind transiently to a complementary nucleic acid domain for 0.1, 1, 5 or 10 seconds.

To achieve transient binding at annealing temperatures, for example, a nucleic acid domain may have a length of 4 to 20 nucleotides. For example, a nucleic acid domain may have a length of 4 to 10, or 4 to 15 nucleotides. In some embodiments, a nucleic acid domain has a length 4, 5, 6, 7, 8, 9, 10, 11, 12, 13, 14, 15, 16, 17, 18, 19 or 20 nucleotides.

In some embodiments, Domains 2A, 2B and 4A of the pro-anchor strand, and Domain 2B of the pro-primer domain, are designed to bind their complementary nucleic acid transiently, at a typical annealing temperature (e.g., 20° C. to 72° C.), to respective complementary nucleic acid domains.

In some embodiments, a nucleic acid domain binds stably to a complementary nucleic acid domain at room temperature. In some embodiments, a nucleic acid domain binds stably to a complementary nucleic acid domain at annealing temperature. In some embodiments, a nucleic acid domain bound stably to a complementary nucleic acid domain binds to the complementary nucleic acid domain for greater than 10 seconds. For example, a nucleic acid domain may bind stably to a complementary nucleic acid domain for at least 15, at least 20, at least 30, at least 40, at least 50, at least 60 seconds, or more. In some embodiments, a nucleic acid domain binds stably to a complementary nucleic acid domain for 1 to 5 minutes.

To achieve stable binding at annealing temperatures(e.g., 20° C. to 72° C.), a nucleic acid domain may have a length of 15 to 100 nucleotides. For example, a nucleic acid domain may have a length of 15 to 90, 15 to 80, 15 to 70, 15 to 60, 15 to 50, 15 to 40, 15 to 30, 20 to 90, 20 to 80, 20 to 70, 20 to 60, 20 to 50, or 20 to 40 nucleotides. In some embodiments, a nucleic acid domain has a length 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 nucleotides.

In some embodiments, Domains 3A+4A (a contiguous stretch containing Domain 3A and Domain 4A) of the pro-anchor strand, and Domains 1A+2A (a contiguous stretch containing Domain 1A and Domain 2A) of the pro-anchor strand, are designed to bind stably, at a typical annealing temperature, to respective complementary nucleic acid domains. It should be understood that the length of each nucleic acid domain of an RA primer may vary depending on the sequence of the target nucleic acid and the strand displacement kinetics required to permit binding of the sensing molecule to the target nucleic acid and subsequent recruitment and activation of the priming module. Thus, a single nucleic acid domain, or a combination of contiguous nucleic acid domains on the same strand, may have a length of 4 to 100 nucleotides, or more. For example, a single nucleic acid domain, or a combination of contiguous nucleic acid domains, may have a length of 4 to 10, 4 to 15, 4 to 20, 4 to 25, 4 to 30, 4 to 35, 4 to 40, 4 to 45, 4 to 50, 4 to 55, 4 to 60, 4 to 65, 4 to 70, 4 to 75, 4 to 80, 4 to 85, 4 to 90, or 4 to 95 nucleotides. In some embodiments, a single nucleic acid domain, or a combination of contiguous nucleic acid domains, may have a length of 15 to 20, 15 to 25, 15 to 30, 15 to 35, 15 to 40, 15 to 45, 15 to 50, 15 to 55, 15 to 60, 15 to 65, 15 to 70, 15 to 75, 15 to 80, 15 to 85, 15 to 90, or 15 to 95 nucleotides.

In some embodiments, the 3′ end of a strand of a sensing module or a priming module comprises a non-extendable nucleotide (e.g., to prevent the polymerization/extension of the 3′ end of the strand). In some embodiments, the non-extendable nucleotide may be a non-naturally occurring nucleotide or a dideoxy nucleotide. Examples of non-extendable nucleotides include, without limitation, isoC, isoG, deoxyuridine, dP, dZ, 3′-deoxyadenosine, 3′-deoxythymidine, 3′-deoxyguanosine, 3′-deoxycytidine, or an otherwise naturally occurring nucleotide inserted in an inverted orientation (such as inverted dT), as well as nucleotides modified with at least one non-nucleotide moiety such as, for example, morpholinos, threose nucleic acids, phosphates, multi-carbon linkers, amino groups, thiol groups, azide groups and/or alkyne groups.

In some embodiments, after extension of a minimal primer by a polymerase, a part of or all of the priming module becomes part of the extension product. When an RA primer is used in multiple cycles of priming (e.g., is used in PCR), this extension product becomes the template for the synthesis of the opposite strand. In this process, it may be desirable that only the minimal primer portion of the RA primer is copied and the rest of the priming module is not copied, for example. This can be accomplished, for example, by separating the minimal primer from the rest of the priming module (linking the minimal primer to the remaining components of the priming module) with a “polymerase-stopping” or a “polymerase-pausing” moiety. Examples of such moieties include, without limitation, non-nucleotide chemical linkers and modified nucleotides that cannot be recognized by the polymerase. Some nucleotides that can be recognized by the polymerase can also serve this purpose. For example, deoxyuridine can be recognized by many polymerases (such as many reverse transcriptases and Taq), but can stop some archaeal DNA polymerases such as Pfu and Vent. As another example, an unnatural base that can be recognized by a polymerase can be used in the absence of the corresponding nucleotide triphosphate, or in the presence of a variant of the corresponding nucleotide triphosphate which, after being incorporated into the growing DNA chain, cannot be further extended. One embodiment uses isoC to separate the minimal primer from the rest of the priming module (or to link the minimal primer to the other components of the priming module), and in the reaction (a) do not provide deoxy-isoG-triphosphate (d(isoG)TP) in the reaction or (b) provide a 2′,3′-dideoxy variant of the isoG triphosphate, or both. In some aspects, a sensing module is describes as comprising a partially double-stranded nucleic acid comprising a first nucleic acid strand bound to a second nucleic acid strand, wherein the first strand comprises (a) a 5′ domain that includes sequence complementary to the priming module, wherein a portion of the sequence of (a) is complementary to and bound to the second strand, thereby forming a double-stranded region, and (b) a 3′ domain that includes sequence complementary to the target nucleic acid, wherein a portion of the sequence of (b) is complementary to and bound to the second strand, thereby forming a double-stranded region.

Similarly, in some aspects, a priming module is describes as comprising a (c) a 5′ domain that includes sequence complementary to the sensing module, wherein a portion of the sequence of (c) is complementary to and bound to the second strand, thereby forming a double-stranded region, and (d) a 3′ domain that includes (or optionally includes) a chemical linker attached to a minimal primer sequence, wherein the minimal primer sequence is complementary to the target nucleic acid and is bound to the second strand, thereby forming a double-stranded region.

In some embodiments, a priming module is described as comprising a (c) a 5′ domain that includes sequence complementary to the sensing module, wherein a portion of the sequence of (c) is complementary to and bound to the second strand, thereby forming a double-stranded region, and (d) a 3′ domain attached to a minimal primer sequence, wherein the minimal primer sequence is complementary to the target nucleic acid and is bound to the second strand, thereby forming a double-stranded region.

A “double-stranded domain” of a nucleic acid refers to a portion of a nucleic acid (e.g., DNA) containing two nucleic acid strands bound to each other. By contrast, a “single-stranded domain” of a nucleic acid refers to a portion of a single strand of the nucleic acid that is unbound to another portion or unbound to another strand. A “partially double-stranded nucleic acid” refers to a nucleic acid that includes at least one double-stranded domain and at least one single-stranded domain. FIG. 4A depicts an example of a priming module having a double-stranded domain (formed by binding of Domains 1B and 5A of the pro-primer strand to Domains 1A and 5B of the anti-primer domain) and a single-stranded domain (formed by Domain 2B of the pro-primer strand). Likewise, FIG. 4A depicts an examples of a sensing module having a double-stranded region (formed by binding of Domains 2A and 3A of the pro-anchor strand with Domains 2B and 3B of the anti-anchor strand) and two single-stranded regions (one formed by Domain 1A of the pro-anchor strand, and one formed by Domain 4 of the pro-anchor strand). The example of the sensing module shown in FIG. 4A may also be described as having two partially double-stranded domains: one at the 5′ end of the molecule, formed by a double-stranded domain containing Domain 2B bound to Domain 2A, leaving Domain 1A single-stranded, and another at the 3′ end of the molecule, formed by a double-stranded domain containing Domain 3B bound to Domain 3A, leaving Domain 4 single-stranded.

“Sequence complementary to the priming module” refers to a nucleotide sequence that is complementary to and binds to the strand of the priming module containing the minimal primer domain (e.g., the pro-primer strand). As an example, Domains 1A and 2A of the pro-anchor strand of the sensing module in FIG. 4A are collectively considered “sequence complementary to the priming module.”

“Sequence complementary to the sensing module” refers to a nucleotide sequence that is complementary to and binds to the strand of the sensing module that that is designed to bind to the target nucleic acid (e.g., the pro-anchor strand). As an example, Domains 2B and 1B of the pro-primer strand of the priming module in FIG. 4A are collectively considered “sequence complementary to the sensing module.”

“Sequence complementary to the target nucleic acid” refers to a nucleotide sequence that is complementary to and capable of binding to a strand of the target nucleic acid. As an example, Domains 3A and 4A of the pro-anchor strand of the sensing module in FIG. 4A are collectively considered “sequence complementary to the target nucleic acid.”

A sensing module of the present disclosure binds (e.g., binds specifically) to a target nucleic acid and, when bound to the target nucleic acid, recruits and activates the priming module. Binding of a sensing module to a target nucleic acid is based on complementarity. Two nucleic acids, or two nucleic acid domains, are “complementary” to one another if they base-pair, or bind, to each other to form a double-stranded nucleic acid molecule via Watson-Crick interactions (also referred to as hybridization). Two nucleic acids, or two nucleic acid domains, are “perfectly complementary” to one another if every nucleotide of one nucleic acid can base-pair with every nucleotide of the other nucleic acid. Herein, complementarity is presumed to be perfectly complementarity unless otherwise indicated. As used herein, two nucleic acids or nucleic acid domains are “partially complementary” to one another when the two domains are not fully complementary but can bind to each other to form an imperfect duplex when the two domains are used, alone or attached to other molecules or moieties. As used herein, an imperfect duplex is nucleic acid duplex disrupted by mismatches, bulges and/or internal loops. As used herein, two nucleic acids or nucleic acid domains have “similar” sequence when more than 74% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%) of the sequence are identical when properly aligned. “Binding,” in the context of nucleic acids, refers to an association between at least nucleic acids, or two nucleic acid domains, due to, for example, electrostatic, hydrophobic, ionic and/or hydrogen-bond interactions under physiological conditions.

The RA primers of the present disclosure function via a series of “strand displacement” reactions mechanism (see, e.g., Yurke et al., Nature 406: 605-608, 2000; and Zhang et al. Nature Chemistry 3: 103-113, 2011, each of which is incorporated by reference herein). “Strand displacement” refers to the mechanism by which two nucleic acid strands with identical sequences, when proximate to a single complementary nucleic acid strand (or segment of a strand), undergo relatively rapid (e.g., timescale<1s) competition for that complement strand, ‘displacing’ each other from the complement presumably by a ‘random-walk’ mechanism.

An example of such a strand displacement reaction using an RA primer is shown in FIG. 5. First, the pro-anchor strand of the sensing module uses Domain 4A to bind a Domain 4B of the target nucleic acid and initiates a strand displacement reaction, which ultimately leads to hybridization between Domains 3A and 4A of the pro-anchor and the Domains 3B and 4B of the target nucleic acid, respectively, as well as dissociation of the anti-anchor strand (FIG. 5, Step 1). The dissociation of the anti-anchor strand of the sensing module exposes Domain 2A of the pro-anchor strand, which in turn can interact with Domain 2B of the pro-primer strand of the priming module to initiate another strand displacement reaction. This leads to hybridization between Domains 1B and 2B of the pro-anchor strand and Domains 1A and 2B of the pro-primer strand, respectively, as well as the dissociation of the anti-primer strand of the priming module (FIG. 5, Step 2). The dissociation of the anti-primer strand of the priming module exposes Domain 6A of the pro-primer strand (i.e., the minimal primer domain), which then binds to Domain 6B of the target nucleic acid and is extended by a polymerase (FIG. 5, Step 3).

In some embodiments, the polymerase used in a reaction has strand-displacement activity (e.g., for Vent polymerase, Bst polymerase and variants thereof). In such embodiments, the polymerizing (extending) 3′ end of the minimal primer domain displaces the incumbent pro-anchor strand (FIG. 5, Step 4). In other embodiments, the polymerase used in a reaction has 5′-to-3′ exonuclease activity (e.g., Taq polymerase). In such embodiments, the polymerase may degrade Domains 3A and 4 of the pro-anchor strand while continuing to catalyze polymerization. In yet other embodiments, the polymerase used in a reaction has neither strand-displacement activity nor 5′-to-3′ exonuclease activity, in which case, the temperature of the reaction may be raised above the melting temperature of the target-specific domains (i.e., Domains 3A and 4A) of the pro-anchor strand so that the target-specific domains dissociate spontaneously from the target nucleic acid. In such embodiments, the gap between the Domains 6B and 3B on the template should be long enough so that the partial extension product following Step 3 (FIG. 5) has a higher melting temperature than the target-specific domains of the pro-anchor.

As shown in FIG. 5, the minimal primer of the pro-primer strand of the priming module binds to the target nucleic acid upstream of the pro-anchor strand of the sensing module. A priming module, or a strand of a priming module, is considered “upstream” relative to a sensing module if, when both modules (or strands thereof) are bound to the target and are in the presence of polymerase and nucleotide triphosphates, the priming module (or strand thereof) is extended in the 5′ to 3′ direction, resulting in a newly formed nucleic acid extension that displaces the sensing module (or strand thereof).

Upon binding of a sensing module to a target nucleic acid, the sensing module “recruits” and “activates” the priming module.

“Recruiting” refers to the process by which the priming module binds to the sensing module. In the example shown in FIGS. 4A and 5, recruiting occurs when dissociation of the anti-anchor strand of the sensing module exposes Domain 2A of the pro-anchor strand, which in turn can interact with Domain 2B of the pro-primer strand of the priming module to initiate another strand displacement reaction. This leads to hybridization between Domains 1B and 2B of the pro-anchor strand and Domains 1A and 2B of the pro-primer strand, respectively, as well as the dissociation of the anti-primer strand of the priming module (FIG. 5, Steps 1-2). Recruitment is complete upon binding of Domains 1B and 2B of the pro-anchor strand to Domains 1A and 2B of the pro-primer strand.

“Activation” refers to the process by which the 3′ end of the pro-primer strand dissociates from the anti-primer strand of priming module and is available to bind to the target nucleic acid. Activation may also refer to the process by which the 3′ end of the minimal primer of the priming module becomes more exposed to the template. In the example of FIGS. 4A and 5, the activation process is the process in which the pro-primer strand dissociates from the anti-primer strand of priming module and is available to bind to the target nucleic acid. In this example, dissociation of the anti-primer strand of the priming module exposes Domain 6A of the pro-primer strand (i.e., the minimal primer domain) (FIG. 5, Steps 2-3). The minimal primer Domain 6A, now “activated,” binds to Domain 6B of the target nucleic acid and is extended by a polymerase.

In some embodiments, a priming module is linked to a sensing molecule, even in the absence of a target nucleic acid. For example, a priming module may be “tethered” to a sensing module through a “passive linker,” which refers to a linker that does not participate in nucleic acid hybridization or strand displacement. A passive linker may be a chemical linker or a nucleic acid linker. The length and location of a passive linker may vary, provided it does not pose steric hindrance to hybridization or strand displacement. For example, a passive linker may link the 5′ end of the pro-anchor strand to the 5′ end of the pro-primer strand. As another example, the passive linker may link the 5′ end of the pro-anchor strand to the 5′ end of the anti-primer strand. The linker may also link the 5′ end of the pro-anchor strand to the 3′ end of the anti-primer strand. Other linkage arrangements are contemplated.

Examples of chemical linkers for used in accordance with the present disclosure (e.g., to link a sensing module to a priming module, or to link a minimal primer domain to other domains of the pro-primer strand) include, without limitation, polyethylene glycol (PEG), hexethylene glycol, an alkyl spacer, a peptide nucleic acid (PNA), or a locked nucleic acid (LNA).

In some embodiments, one domain may be linked to another domain, or the priming module may be linked to the sensing module, using a chemical conjugation reaction (e.g., “click chemistry”). For example, the two domains (or modules via domains) may be linked to each other using an azide alkyne Huisgen cycloaddition reaction (Rostov, stev, V. V., et al. Angewandte Chemie International Edition 41 (14): 2596-2599, 2002; Tornoe, C. W. et al. Journal of Organic Chemistry 67 (9): 3057-3064, 2002). In some embodiments, two domains (or modules via domains) may be linked to each other using other conjugation reactions involving amine, carboxyl, sulfhydryl, or carbonyl groups, or a combination of any of the foregoing reactions.

As used herein, a “primer” serves as a starting point for nucleic acid (e.g., DNA) synthesis. Thus, the RA primers of the present disclosure typically serve as the starting point for nucleic acid synthesis and may be used in a variety of applications that involve nucleic acid synthesis. Examples of such amplification processes contemplated by the present disclosure include isothermal DNA amplification, including transcription-mediated amplification (TMA), nucleic acid sequence based amplification (NASBA), strand displacement amplification (SDA) and loop-mediated isothermal amplification (LAMP). Non-amplification processes are also contemplated herein. For example, the RA primers may be used in processes such as reverse transcription.

A target nucleic acid (e.g., DNA or RNA) of interest may be any nucleic acid of interest. A target nucleic acid may be a single-stranded (ss) or double-stranded (ds) nucleic acid. In some embodiments, a target nucleic acid is a rare allele. An “allele” is one of a number of alternative forms of the same gene or same genetic locus. Alleles may differ from each other by a single nucleotide in the form of alteration, insertion or deletion. A “wild-type allele” refers to the major (more or most common) allele in a given plurality of nucleic acids. Conversely, a “rare allele,” refers to the minor (less or least common) allele in the same plurality of nucleic acids. For example, in some embodiments, a plurality of nucleic acids encoding gene X may contain 10- to 1,000,000-fold, or 100- to 1,000,000-fold, more of allele X_{A} than allele X_{B}, where allele X_{A} and allele X_{B} differ by a single nucleotide. Allele X_{A} is considered to be the “wild-type allele,” while allele X_{B} is considered to be the “rare allele.”

Target nucleic acids may be, for example, DNA, RNA, or the DNA product of RNA subjected to reverse transcription. In some embodiments, a target nucleic acid may be a mixture (chimera) of DNA and RNA. In some embodiments, a target nucleic acid may comprise artificial nucleic acid analogs, for example, peptide nucleic acids (Nielsen et al. Science 254(5037): 1497-500 (1991)) or locked nucleic acids (Alexei et al. Tetrahedron 54(14): 3607-30 (1998)). In some embodiments, a target nucleic acid may be naturally occurring (e.g., genomic DNA) or it may be synthetic (e.g., from a genomic library). As used herein, a “naturally occurring” nucleic acid sequence is a sequence that is present in nucleic acid molecules of organisms or viruses that exist in nature in the absence of human intervention. In some embodiments, a target nucleic acid is genomic DNA, messenger RNA, ribosomal RNA, micro-RNA, pre-micro-RNA, pri-micro-RNA, viral DNA, viral RNA or piwi-RNA. In some embodiments, a target nucleic acid is a nucleic acid that naturally occurs in an organism or virus. In some embodiments a target nucleic acid is the nucleic acid of a pathogenic organism or virus. In some embodiments, the presence or absence of a target nucleic acid in a subject is indicative that the subject has a disease or disorder or is predisposed to acquire a disease or disorder. In some embodiments, the presence or absence of a target nucleic acid in a subject is indicative that the subject will respond well or poorly to a treatment, such as a drug, to treat a disease or disorder.

The term nucleic acid refers to a polymeric form of nucleotides of any length, either deoxyribonucleotides or ribonucleotides, or analogs thereof. Nucleic acids may have any three-dimensional structure, and may perform any function. The following are non-limiting examples of nucleic acids: coding or non-coding regions of a gene or gene fragment, loci (locus) defined from linkage analysis, exons, introns, messenger RNA (mRNA), transfer RNA, ribosomal RNA, ribozymes, cDNA, recombinant nucleic acids, branched nucleic acids, plasmids, vectors, isolated DNA of any sequence, isolated RNA of any sequence, nucleic acid probes, and primers. A nucleic acid may comprise modified nucleotides, such as methylated nucleotides and nucleotide analogs. A nucleic acid may be further modified, such as by conjugation with a labeling component.

A target nucleic acid utilized herein can be any nucleic acid, for example, human nucleic acids, bacterial nucleic acids, or viral nucleic acids. A target nucleic acid sample can be, for example, a nucleic acid sample from one or more cells, tissues, or bodily fluids. Target samples can be derived from any source including, but not limited to, eukaryotes, plants, animals, vertebrates, fish, mammals, humans, non-humans, bacteria, microbes, viruses, biological sources, serum, plasma, blood, urine, semen, lymphatic fluid, cerebrospinal fluid, amniotic fluid, biopsies, needle aspiration biopsies, cancers, tumors, tissues, cells, cell lysates, crude cell lysates, tissue lysates, tissue culture cells, buccal swabs, mouthwashes, stool, mummified tissue, forensic sources, autopsies, archeological sources, infections, nosocomial infections, production sources, drug preparations, biological molecule productions, protein preparations, lipid preparations, carbohydrate preparations, inanimate objects, air, soil, sap, metal, fossils, excavated materials, and/or other terrestrial or extra-terrestrial materials and sources. The sample may also contain mixtures of material from one source or different sources. For example, nucleic acids of an infecting bacterium or virus can be amplified along with human nucleic acids when nucleic acids from such infected cells or tissues are amplified using the disclosed methods. Types of useful target samples include eukaryotic samples, plant samples, animal samples, vertebrate samples, fish samples, mammalian samples, human samples, non-human samples, bacterial samples, microbial samples, viral samples, biological samples, serum samples, plasma samples, blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, crude cell lysate samples, tissue lysate samples, tissue culture cell samples, buccal swab samples, mouthwash samples, stool samples, mummified tissue samples, autopsy samples, archeological samples, infection samples, nosocomial infection samples, production samples, drug preparation samples, biological molecule production samples, protein preparation samples, lipid preparation samples, carbohydrate preparation samples, inanimate object samples, air samples, soil samples, sap samples, metal samples, fossil samples, excavated material samples, and/or other terrestrial or extra-terrestrial samples.

In some embodiments, a target nucleic acid utilized as provided herein comprises repetitive sequence, secondary structure, and/or a high G/C content.

In some embodiments, a target nucleic acid is about 100 to about 1,000,000 nucleotides (nt) or base pairs (bp) in length. In some embodiments, the target and/or pseudo-target nucleic acid is about 100 to about 1000, about 1000 to about 10,000, about 10,000 to about 100,000, or about 100,000 to about 1,000,000 nucleotides in length. In some embodiments, the target and/or pseudo-target nucleic acid is about 100, about 200, about 300, about 400, about 500, about 600, about 700, about 800, about 900, about 1,000, about 2,000, about 3,000, about 4,000, about 5,000, about 6,000, about 7,000, about 8,000, about 9000, about 10,000, about 20,000, about 30,000, about 40,000, about 50,000, about 60,000, about 70,000, about 80,000, about 90,000, about 100,000, about 200,000, about 300,000, about 400,000, about 500,000, about 600,000, about 700,000, about 800,000, about 900,000, or about 1,000,000 nucleotides in length. It is to be understood that a target nucleic acid may be provided in the context of a longer nucleic acid (e.g., such as a coding sequence or gene within a chromosome or a chromosome fragment).

In some embodiments, a target nucleic acid is linear, while in other embodiments, a target nucleic acid is circular (e.g., plasmid DNA, mitochondrial DNA, or plastid DNA).

Partially Double-Stranded Blocker Primers (dsBlockers)

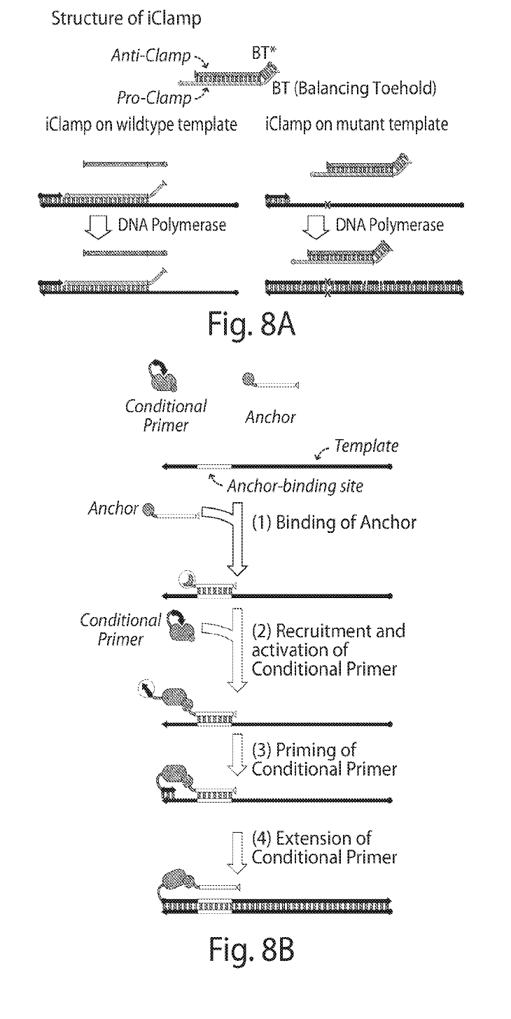

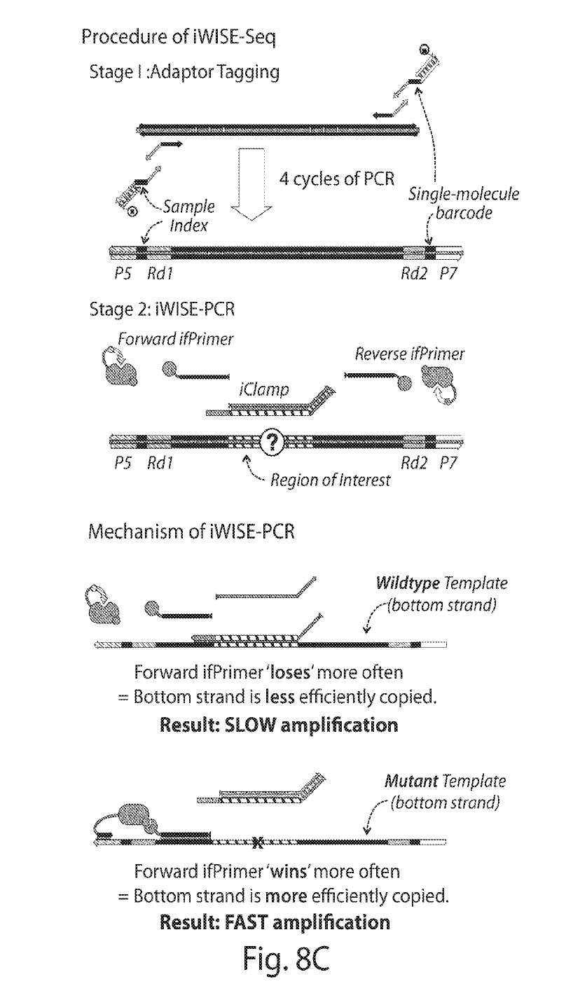

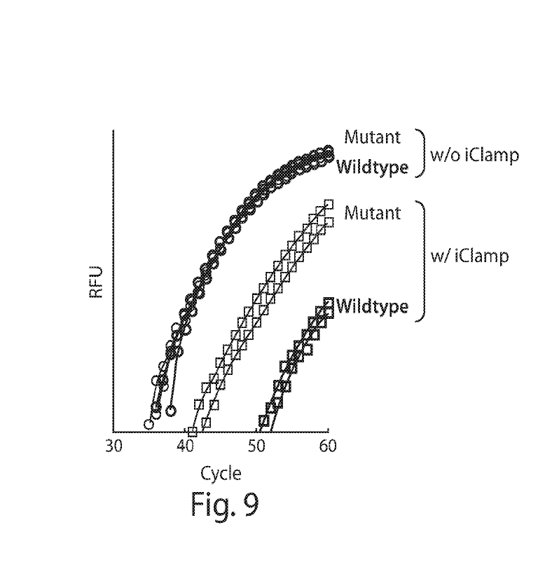

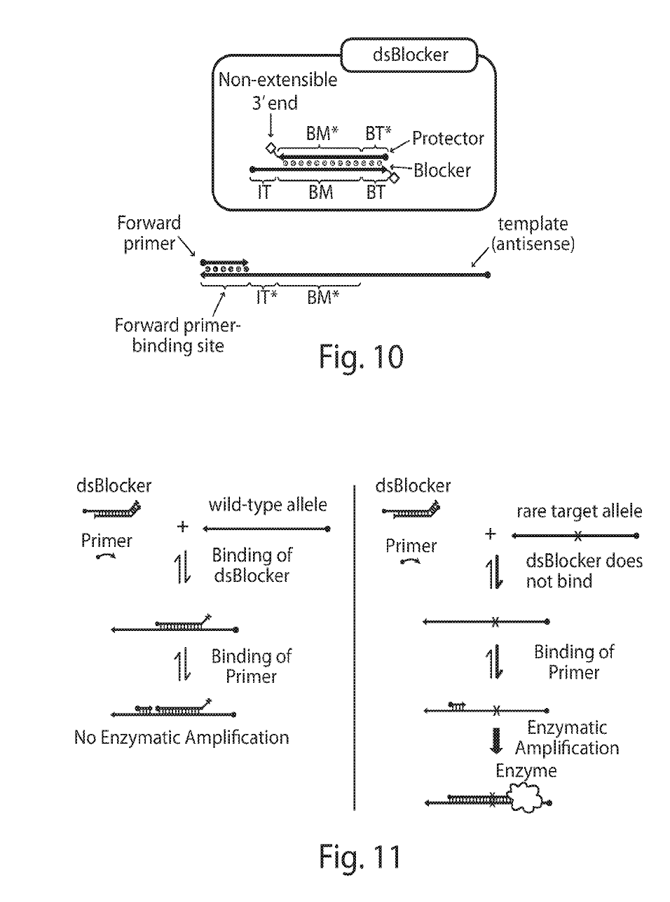

In some embodiments, biased amplification of a target nucleic is achieved by engineering a set of nucleic acids that collectively function to block amplification of non-target nucleic acid. As used herein, a “dsBlocker” of the present disclosure refers to an engineered partially double-stranded nucleic acid that comprises first (“blocker”) and second (“protector”) nucleic acid strands arranged into (i) one double-stranded pseudo-target non-specific domain (e.g., “BT”/“BT*”), (ii) one double-stranded pseudo-target specific domain (e.g., “BM”/“BM*”), and (ii) one single-stranded pseudo-target specific domain (“IT)” contributed to by the first nucleic acid strand, wherein the double-stranded pseudo-target non-specific domain has a standard free energy (AG) approximately equal to the standard free energy for the single-stranded pseudo-target specific domain bound to a rare target nucleic acid, and wherein the 3′ end of the first nucleic acid strand and the 3′ of the second nucleic acid strand are non-extendable (FIG. 10). A “dsBlocker” is also referred to herein as an “iClamp” primer, as depicted in FIG. 8A.

FIG. 10 shows an example of a dsBlocker having blocker and protector strand. The blocker strand may be divided into three domains (ordered 5′ to 3′): the initial toehold domain (“IT”), the branch-migration domain (“BM”), and the balancing toehold domain (“BT”). The protector strand may be divided into two domains (ordered 5′ to 3′): the balancing toehold domain (“BT*”), which is complementary to the balancing toehold domain (“BT”) of the blocker strand, and the branch migration domain (“BM*”), which is complementary to the branch migration domain (“BM”) of the blocker strand. In some embodiments, the blocker strand is a contiguous nucleic acid. In some embodiments, the protector strand is a contiguous nucleic acid.

In some embodiments, the position of the initial toehold domain and the balancing toehold domain can be interchanged such that the initial toehold domain is located at the 3′ end of the blocker strand and the balancing toehold domain is located at the 5′ end of the blocker strand. In such embodiments, the BT* domain is located at the 3′ end of the protector strand.

As shown in FIG. 11, when a dsBlocker designed to bind to a wild-type allele contacts a wild-type allele, the two strands of the dsBlocker dissociate as the strand that is complementary to the wild-type allele binds to a strand of the wild-type allele. By contrast, when the dsBlocker contacts a target allele, dissociation of the two strands of the dsBlocker is not favored, and the dsBlocker does not bind to the target allele. Thus, a nondiscriminatory primer binds to and is extended along the length of a strand of the target allele, resulting in preferential amplification of the target allele.

In some embodiments, dsBlocker nucleic acid amplification (nucleic acid synthesis/amplification using a dsBlocker) may be used to amplify a target allele (or other nucleic acid). This is achieved by blocking amplification of the wild-type allele. In such embodiments, the IT-BM domains of the blocker strand may be engineered to be complementary to the wild-type allele (FIG. 11). The sequence of the BT domain may be designed according to several parameters to avoid unwanted hybridization. For example, the dsBlocker can be designed in the following processes.

Step 1. Choose the annealing temperature of the reaction. The annealing temperature should be high enough so that the pseudo-target strand does not form extensive secondary structure, but low enough so that typical primers have sufficient affinity to the primer-binding sites on the template. The annealing temperature is typically in the range of 55° C. to 70° C.

Step 2. Design IT and BM so that (a) the region on the pseudo-target strand that binds IT or BM encompasses the polymorphism site; (b) IT binds the pseudo-target strand weakly at the annealing temperature; (c) the IT-BM region of the blocker strand binds the pseudo-target strand stably 5° C. above the annealing temperature; (d) BM binds the pseudo-target strand weakly 10° C. above the annealing temperature.

Step 3. Generate a random sequence as candidate of BT so that the candidate BT binds its complementary strand with similar affinity as IT binds its complementary strand. For example, BT may have similar length and GC content as IT.

Step 4. Examine in silico whether BT erroneously binds the pseudo-target strand or the IT-BM region of the blocker strand. If so, repeat from Step 3. If not, use the candidate BT sequence.

The performance of dsBlocker can be further optimized by adjusting the length and/or GC content of the BT and/or BT* domains.

It is contemplated herein that the interaction between BT and BT* may be replaced by other forms of pseudo-target non-specific interactions, including indirect hybridization, a mixture of direct and indirect hybridization, protein-protein interaction, protein-small molecule interaction, magnetic interaction, electronic charge interaction, and the like.

In some embodiments, the free energy (ΔG) of binding between the BT domain and the BT* domain may be ˜2 kcal/mole, or ˜1 kcal/mole, higher than the ΔG of binding between the IT domain to non-target allele (or other nucleic acid, or non-target allele, e.g., wild-type allele) at annealing temperature (i.e., IT domain: non-target nucleic acid binding is stronger than BT domain:BT* domain binding). As a result, the ΔG of the blocker strand binding to the protector strand is ˜2 kcal/mole, or ˜1 kcal/mole, higher than the ΔG of the blocker strand binding to the non-target nucleic acid (i.e., the blocker: non-target nucleic acid binding is stronger than the blocker:protector binding). A single-nucleotide change (e.g., mutation, insertion or deletion) in the target nucleic acid may destabilize the blocker:target strand duplex by ˜2 kcal/mole. Thus, when the dsBlocker of the present disclosure contacts non-target nucleic acid, the non-target nucleic acid (e.g., more than 50% of the non-target nucleic acid) displaces the protector strand and binds to the blocker strand (e.g., at equilibrium). By comparison, when the dsBlocker contacts the target nucleic acid, which contains a mutation in the region complementary to the IT-BM domains of the blocker strand, only a small fraction of the target nucleic acid (e.g., less than 50% of the target nucleic acid) binds to the blocker strand because the target nucleic acid strand binds the Blocker strand less well than the blocker strand binding to the protector strand (e.g., displaces the protector strand and binds to the blocker strand). The single-nucleotide change (e.g., mutation, insertion or deletion) in the target nucleic acid typically destabilizes the blocker: target nucleic acid duplex by ˜1 to ˜4 kcal/mole. Thus, complementary binding between the blocker strand and the protector strand may be preferred over binding between the blocker strand and the target nucleic acid, where there is at least one nucleotide difference.

In some embodiments, the blocker and/or the protector strand comprises a non-extendable nucleotide at its 3′ end. In some embodiments, the blocker and/or the protector strand comprises a nucleotide that blocks the addition of more nucleotides to the 3′ end. In some embodiments, the blocker and/or the protector strand comprises a nucleotide that blocks the degradation of the 3′ end. In some embodiments, the non-extendable nucleotide is a non-naturally occurring nucleotide or a dideoxy nucleotide. In some embodiments, the non-naturally occurring nucleotide is isoC, isoG or deoxyuridine, 3′-deoxyadenosine, 3′-deoxythymidine, 3′-deoxyguanosine, 3′-deoxycytidine and the like, or otherwise naturally occurring nucleotide inserted in an inverted orientation.

In some embodiments, methods of the present disclosure comprise contacting a pool of target and non-target nucleic acids, such as wild-type alleles, with (a) single-stranded primer, which is engineered to be complementary to a target nucleic acid of interest and, in some embodiments, to a non-target nucleic acid (or pseudo-target nucleic acid), and (b) a dsBlocker, and extending the engineered single-stranded primer at its 3′ end in a target-complementary manner in the presence of a polymerase. The blocker strand of the dsBlocker, which is engineered to be complementary to the wild-type nucleic acid (e.g., wild-type allele), preferentially binds to the wild-type nucleic acid, thereby blocking extension of the single-stranded primer. In the same reaction, the single-stranded primer binds to the target nucleic acid and, without being blocked by the blocker strand, is extended. Thus, the target nucleic acid is preferentially amplified.

As discussed above, the blocker strand of the dsBlocker of the present disclosure comprise at least three domains, including an initial toehold (IT) domain, a branch-migration (BM) domain, and a balancing toehold (BT) domain. The initial toehold domain and the branch migration domain have nucleic acid sequences that are complementary to nucleic acid sequences of the pseudo-target nucleic acid. The initial toehold domain and the branch migration domain are therefore able to base-pair with and thus form a complex with a sequence of a pseudo-target nucleic acid when the dsBlocker is contacted with a pseudo-target nucleic acid under appropriate hybridization conditions. The balancing toehold domain is rationally designed, and thus, the sequence of the balancing toehold domain is not designed to be complementary to a sequence in the pseudo-target nucleic acid sequence.

An initial toehold domain is complementary to (and thus hybridizes to) a sequence in the pseudo-target nucleic acid; however, an initial toehold domain does not hybridize to a protector strand. Thus, when the blocker strand is hybridized to the protector strand, the initial toehold domain may also hybridize to the target nucleic acid and/or the pseudo-target nucleic acid. An initial toehold domain may be positioned at the 3′ end or the 5′ end of the blocker strand (e.g., is an extension of the 3′ end or 5′ end of the blocker strand).

In some embodiments, such as in PCR, the primer is engineered so that, when the extension is stopped by the blocker strand, the partial extension product binds the pseudo-target template with a melting temperature at least 0.1° C. lower than the melting temperature of the complex formed by the pseudo-target strand and the blocker strand, so that during a process that the temperature is raised to a higher temperature (e.g., extension temperature and denaturing temperature), the partial extension product dissociates earlier than the blocker strand. It is to be understood that a DNA polymerase may extend a primer on a template at a temperature below the designated extension temperature, such as the annealing temperature.

In some embodiments, there is no gap on the template between the primer-binding site and the blocker strand-binding site. In such embodiments, the primer itself can be considered the partial extension product.

In some embodiments, the polymerase is a DNA polymerase with weak or no strand-displacement activity and no 5′-to-3′ exonuclease activity (e.g., Pfu and PHUSION).

In some embodiments, the polymerase is a DNA polymerase with reported strand-displacement activity or reported 5′-to-3′ exonuclease activity. In such embodiments, the enrichment may still be achieved if the polymerase does not completely displace or degrade the blocker strand during each step of PCR.

In some embodiments, the polymerase is a DNA polymerase with 3′-to-5′ exonuclease activity (also known as the proofreading activity). In some embodiments, the polymerase is a high-fidelity DNA polymerase.

In some embodiments, an initial toehold domain is about 4 nucleotides to about 20 nucleotides in length, about 4 nucleotides to about 15 nucleotides in length, or about 4 nucleotides to about 10 nucleotides in length. In some embodiments, an initial toehold domain is 4, 5, 6, 7, 8, 9, 10, 11, 12, 13, 14, 15, 16, 17, 18, 19 or 20 nucleotides in length. In some embodiments, an initial toehold domain is greater than 20 nucleotides in length, including for example less than or about 25, 30, 35, 40, 45, 50, 55, 60, 65, 70, 75, 80, 85, 90, 95 100 or more nucleotides.

The branch migration domain is complementary to a sequence in the pseudo-target nucleic acid and to a sequence in the protector strand. Thus, when the blocker strand hybridizes to a pseudo-target nucleic acid, the branch migration domain hybridizes to the pseudo-target nucleic acid. When the blocker strand hybridizes to its protector strand, the blocker branch migration (BM) domain hybridizes to the protector branch migration (BM*) domain.

In some embodiments, a branch migration domain is no more than 200, 100, 75, 50, 40, 30, 25 or 20 nucleotides in length. In some embodiments, a branch migration domain is about 10 nucleotides to about 200 nucleotides in length. In some embodiments, a branch migration domain is about 10 nucleotides to about 150 nucleotides, about 10 nucleotides to about 100 nucleotides, or about 10 nucleotides to about 50 nucleotides in length. In some embodiments, a branch migration domain is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 nucleotides in length. In some embodiments, a branch migration domain may be more than 200 nucleotides in length, depending on the pseudo-target nucleic acid.

The balancing toehold domain of a blocker strand and a protector strand are complementary to each other (i.e., form a double-stranded nucleic acid) but are non-complementary to the pseudo-target nucleic acid (i.e., neither forms a double-stranded nucleic acid with the pseudo-target). Thus, when a blocker strand hybridizes to a pseudo-target nucleic acid, the blocker balancing toehold domain does not hybridize to the pseudo-target nucleic acid. When the blocker strand hybridizes to its protector strand, the blocker balancing toehold (BT) domain hybridizes to the protector balancing toehold domain (BT*).

The design of the balancing toehold domain is dependent on the design of the initial toehold domain. In some embodiments, the balancing toehold domain is designed such that the thermodynamic profile of the balancing toehold domain is comparable to that of the initial toehold domain. In some embodiments, the thermodynamic profile is based on a theoretic model, using for example, Mfold software available at the bioinfo website of Rensselaer Polytechnic Institute (RPI). The number and/or nature of nucleotides within a balancing toehold domain is comparable to that of the initial toehold domain. For example, if an initial toehold domain is comprised of about 40% A and T nucleotides and 60% G and C nucleotides, then the balancing toehold domain should also be comprised of about 40% A and T nucleotides and 60% G and C nucleotides. In some embodiments, the balancing toehold domain is designed such that no more than three consecutive nucleotides are complementary to a sequence on the pseudo-target nucleic acid to avoid binding of the balancing toehold domain to the pseudo-target nucleic acid.

In some embodiments, the length of a balancing toehold domain is short enough so that the blocker and protector spontaneously dissociate from each other. In some embodiments, a balancing toehold domain is about 4 nucleotides to about 20 nucleotides in length, about 4 nucleotides to about 15 nucleotides in length, or about 4 nucleotides to about 10 nucleotides in length. In some embodiments, a balancing toehold domain is 4, 5, 6, 7, 8, 9, 10, 11, 12, 13, 14, 15, 16, 17, 18, 19 or 20 nucleotides in length. In some embodiments, a balancing toehold domain is greater than 20 nucleotides, including for example less than about 25, 30, 35, 40, 45, 50, 55, 60, 65, 70, 75, 80, 85, 90, 95, 100 or more nucleotides. In some embodiments, the number of consecutive nucleotides that are complementary to a nucleotide sequence within the pseudo-target nucleic acid may be greater than three provided that the balancing toehold domain does not bind to the pseudo-target nucleic acid.

In some embodiments, the design of a balancing toehold domain does not depend on the concentration of the dsBlocker or the temperature at which the dsBlocker is formed/used. In some embodiments, a balancing toehold domain is designed such that the standard free energy for the reaction in which the protector strand is displaced from the blocker strand by the pseudo-target nucleic acid is close to zero kcal/mol. As used herein, “close to zero” means the standard free energy for the reaction is within 3.5 kcal/mol from 0 kcal/mol. In some embodiments, the standard free energy of this displacement reaction is within 3.5, 3.0, 2.5, 2.0, 0.9, 0.8, 0.7, 0.6, 0.5, 0.4, 0.3, 0.2, or 0.1 kcal/mol of zero kcal/mol.

In some embodiments, the design of a balancing toehold domain will be dependent on the dsBlocker concentration as well as reaction temperature. In such embodiments, a balancing toehold domain is designed so that the standard free energy for the reaction in which the protector strand is displaced from the blocker strand by the pseudo-target nucleic acid plus RT ln(c) is close to zero kcal/mol, where R is the universal gas constant (0.0019858775(34) kcal/mol·K), T is the temperature at which the dsBlocker is used, and c is the concentration at which dsBlocker is used. In some embodiments, the temperature at which the dsBlocker is used is about 273 K (0° C.), 277 K, 283 K, 288 K, 293K, 298 K, 303 K, 308 K, 313 K, 318 K, 323 K, 328 K, 333 K, 338 K, 343 K, 348 K, 353 K, 358 K or 363 K (90° C.). In some embodiments, the concentration (c) at which the dsBlocker is used is about 1 nM, 2 nM, 3 nM, 4 nM, 5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 125 nM, 150 nM, 175 nM, 200 nM, 225 nM, 250 nM, 300 nM, 350 nM, 400 nM, 450 nM, 500 nM, 600 nM, 700 nM, 800 nM, 900 nM or 1 μM. In some embodiments, the standard free energy of this displacement reaction plus RT ln(c) is within 3.5, 3.0, 2.5, 2.0, 0.9, 0.8, 0.7, 0.6, 0.5, 0.4, 0.3, 0.2 or 0.1 kcal/mol of zero kcal/mol.

In some embodiments, a dsBlocker may include one or more hairpin domains that connect the blocker strand to the protector strand. In some embodiments, the hairpin domain of a dsBlocker can be of any length. In some embodiments, the hairpin domain is more than 30, 25, 20, 19, 18, 17, 16, 15, 14, 13, 12, 11, 10, 9, 8, 7, 6, 5, 4 or 3 nucleotides in length. In some embodiments, the sequence of the hairpin is not complementary to a sequence of the pseudo-target nucleic acid.

In some embodiments, the hairpin domain has a poly-mononucleotide sequence, such as a poly-adenosine sequence, poly-deoxyadenosine sequence, a poly-5′-methyluridine sequence, a poly-thymidine sequence, a poly-guanosine sequence, a poly-deoxyguanosine sequence, a poly-cytidine sequence, a poly-deoxycytidine sequence, a poly-uridine sequence or a poly-deoxyuridine sequence. In some embodiments, the hairpin loop is or contains a chemical linker. In some embodiments, the chemical linker is polyethylene glycol, an alkyl spacer, a PNA or a LNA.

A dsBlocker of the present disclosure may be one of at least two orientations. For example, in one orientation, the initial toehold domain is located at the 5′ end, immediately adjacent to the blocker branch migration domain (i.e., no intervening nucleotides between the two domains), and the blocker balancing toehold domain is located at the 3′ end, immediately adjacent to the blocker branch migration domain. In this orientation, the protector balancing toehold domain is at the 5′ end of the protector strand, immediately adjacent to the protector branch migration domain. A nucleic acid sequence, domain or region is “immediately adjacent to,” “immediately 5′” or “immediately 3′” to another sequence if the two sequences are part of the same nucleic acid and if no bases separate the two sequences. In another orientation, the initial toehold domain is located at the 3′ end, immediately adjacent to the blocker branch migration domain, and the blocker toehold balancing domain is located at the 5′ end, immediately adjacent to the blocker branch migration domain. In this orientation, the protector balancing toehold domain is at the 3′ end of the protector strand, immediately adjacent to the protector branch migration domain.

In some embodiments, a dsBlocker comprises a blocker strand longer than the protector strand, the difference in length being dependent on the length of the initial toehold domain of the blocker strand. The lengths of the primers are designed such that hybridization of the blocker strand to the pseudo-target nucleic acid has a standard free energy (ΔG°) close to zero. Release of the protector strand (from the dsBlocker) ensures that this hybridization reaction is entropically near-neutral and robust to concentration. As a result, in some embodiments, this reaction at room temperature (e.g., about 25° C. or about 298 K) parallels the specificity of hybridization achieved at near melting temperature across many conditions.

As intended herein, a ΔG° (change in standard free energy) “close to zero” refers to an absolute value (amount) less than or about 1 kcal/mol, less than or about 2 kcal/mol, less than or about 3 kcal/mol, or less than or about 3.5 kcal/mol. In some embodiments, the standard free energy of a balancing toehold domain or initial toehold domain is >−1 kcal/mol to <1 kcal/mol>−3 kcal/mol to <3 kcal/mol or >−3.5 kcal/mol to <3.5 kcal/mol.

dsBlockers of the present disclosure may be prepared at a ratio of protector strand to blocker strand of about 2:1 to about 5:1, or 1:1 to about 5:1. In some embodiments, the ratio of protector strand to blocker strand is about 1:1, about 2:1, about 3:1, about 4:1, or about 5:1. In some embodiments, the ratio of protector strand to blocker strand is 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.2:1, 4.3:1, 4.4:1, 4.5:1, 4.6:1, 4.7:1, 4.8:1, 4.9:1, or 5:1. The dsBlockers may also be used together with excess protector strand in any of the assays or reactions described herein. The protector strand may be in about equal to or more than 1.01-, 2-, 5-, 10-, 20-, 50-, 100-, or 500-fold molar excess relative to the primer (e.g., blocker strand).

Simulating Target Nucleic Acid (e.g., Rare Allele) Enrichment Using dsBlocker in PCR.

Methods of predicting thermodynamics of nucleic hybridization, and dynamic programming algorithms for computing minimum free energy (MFE) structure and partition function, are well developed (SantaLucia, J., et al. Annu Rev Biophys Biomol Struct, 33:415-440, 2004; Dirks, R. M., et al. SIAM Rev, 49(1):65-88, 2007). For a given hybridization reaction, the standard Gibson free energy change at a given temperature can be calculated using the equation ΔG°=ΔH°−T·ΔS°. With this knowledge, several publicly available software programs (e.g., HyTher, Mfold, UNAfold and NUPACK) can be used to predict the ΔG° and equilibrium concentration of each nucleic acid strand among a plurality (e.g., mixture/combination) of nucleic acids.

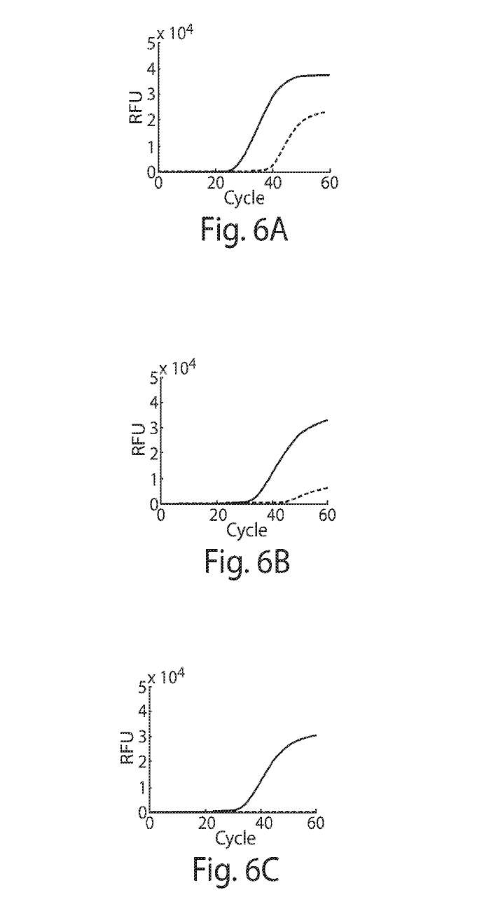

In the embodiments where the pseudo-target non-specific interaction pair is a pair of complementary oligonucleotides (FIG. 6A), the performance of the dsBlocker can be estimated using the following procedure. Without being bound by theory, given the buffer condition and the reaction temperature, the equilibrium concentrations of all possible hybridization products and intermediates for a nucleic acid hybridization reactions that involve the dsBlocker can be calculated using the predicted ΔG° values for all interactions. For example, for hybridization between a pseudo-target template nucleic acid and the blocker strand, at equilibrium:

$$\frac{[ template \text{:} blocker strand ] ( 1 M )}{[ template ] [ blocker strand ]} = {e}^{- Δ G° / RT} \qquad ( 4 )$$

where [template:blocker strand], [template] and [blocker strand] are equilibrium concentrations; T is the annealing temperature (Kelvin temperature), and ΔG° is the standard free energy change of the hybridization reaction.

For a reaction mixture containing multiple strands, the ΔG° value for each pair of partially or fully complementary strands can be predicted, thus an equation that governs the ratios among equilibrium concentration, such as equation (4), can be established. This set of equations plus the equations that reflect conservation of material establish an equation set that has a unique solution. The solution can be computed either analytically or numerically.

At the exponential phase of a nucleic acid amplification reaction (e.g., polymerase chain reaction (PCR)), the concentration of template (either target or pseudo-target) is very low (<5% of the concentration of the blocker strand, or <1% of the concentration of the blocker strand). The ratio of [template]/[template:blocker] is independent of the template concentration and is consistent in each thermocycle of a nucleic acid amplification reaction. Additionally, when a primer is designed to bind to the region on the template downstream of the region on the template that the blocker strand binds, the primer cannot be fully extended if the blocker strand of the dsBlocker is bound to the template. Thus, the template strands that are not bound by the blocker strand of the dsBlocker are assumed to be copied into the complementary strand by the primer and the polymerase; whereas the template strands that are bound by the blocker strand of the dsBlocker are assumed to be not copied into the complementary strand by the primer and the polymerase. The extension efficiency (EE) of an amplification cycle, defined as the fraction of a template that is copied by a primer in the cycle of the exponential amplification reaction, can be calculated using the following equation:

$$EE = [ template ] / ( [ template ] + [ template \text{:} blocker strand ] ) = \frac{[ template ] / [ template \text{:} blocker strand ]}{[ template ] / [ template \text{:} blocker strand ] + 1} \qquad ( 5 )$$

When the component of a dsBlocker nucleic acid amplification reaction, the sequence and concentration of each component, the salinity and the annealing temperature are specified, the EE value (which is constant for each round of exponential amplification) can be calculated.

The single-stranded primer that primes the pseudo-target template strand may be referred to a forward primer, and this forward primer is blocked when the pseudo-target template strand is bound by the blocker strand of a dsBlocker. The pseudo-target template strand that binds the blocker strand of the dsBlocker may be referred to as the antisense strand. The primer that binds the antisense strand may be referred to a forward primer, and this forward primer is blocked when the antisense strand is bound by the blocker strand of a dsBlocker. The pseudo-target template strand that is complementary the antisense strand may be referred to as the sense strand. A primer that is extended on the sense strand may be referred to as a reverse primer. Even though the protector strand of a dsBlocker can hybridize to the sense strand of the pseudo-target template, this hybridization is designed to be unstable at the annealing or extension temperature.

The following definitions apply:

(1) S(0) and AS(0) are the initial concentrations of the sense strand and the antisense strand of the pseudo-target template, respectively;

(2) S(n) and AS(n) (nεN⁺) are the concentration of the sense strand and the antisense strand after the nth cycle.

Thus:

S(n)=S(n−1)+AS(n−1)·EE (6a)

AS(n)=AS(n−1)+S(n−1)·1 (6b)

‘Fold-amplification’ after n cycles is defined as [S(n)+AS(n)]/[S(0)+AS(0)].

The above theories may be used to compare the potential performance of dsBlocker nucleic acid amplification of the present disclosure and traditional wild type-blocking PCR (Dominguez, P. L. et al. Oncogene, 24(45): 6830-4, 2005). The following set of sequences is used as an example:

The antisense strand of pseudo-target template comprises a sequence of 5′-ttcatcagtgatcaccgcccATCCGACGCTATTTGTGCCG[A]TATCTAAGCctattgagtatttc-3′ (SEQ ID NO:21). The antisense strand of rare target template comprises a sequence of 5′-ttcatcagtgatcaccgcccATCCGACGCTATTTGTGCCG[C]TATCTAAGC ctattgagtatttc-3′ (SEQ ID NO:22). For both sequences, the region that can hybridize to the blocker strand of the dsBlocker is shown in upper case letters, and the base that varies between the pseudo-target and target is enclosed by brackets.

Traditional Wild Type-Blocking PCR.

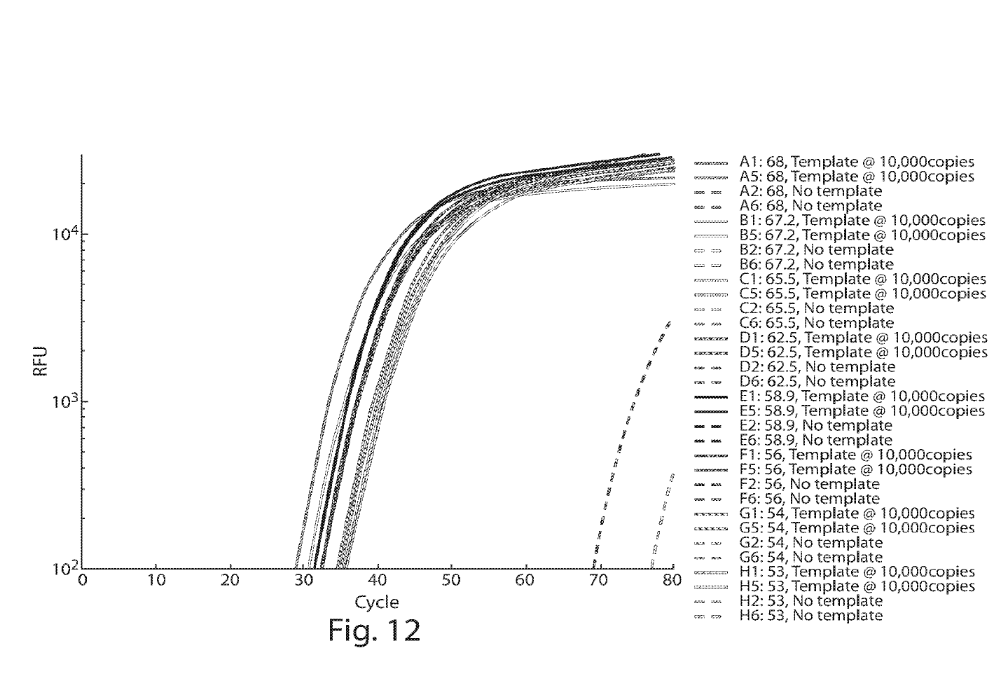

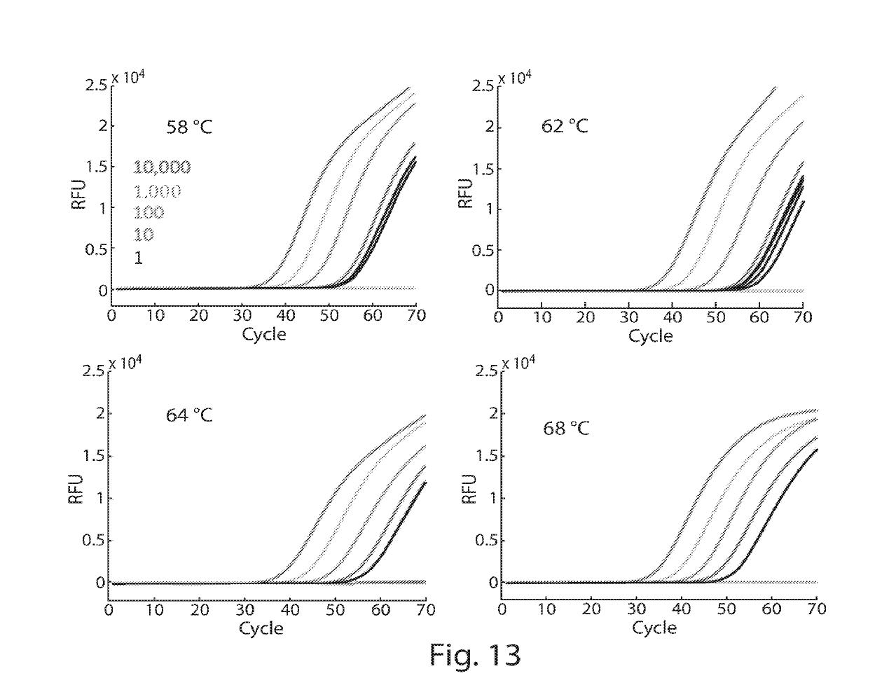

A single-stranded oligonucleotide blocker (also known as ‘clamp’) was designed to hybridize to the antisense strand of pseudo-target template to block extension of the forward primer. The sequence of single-stranded oligonucleotide blocker is as follows: 5′-GCTTAGATA[T]CGGCAC AAATAGCGTCGGAT-3′ (SEQ ID NO:23), where the nucleotide that differentiates the pseudo-target and the target template is enclosed by brackets. The concentration of the single-stranded oligonucleotide blocker was set to be 100 nM. Using established thermodynamic parameters (SantaLucia, J., et al. 2004; Dirks, R. M., et al. 2007) at a salinity of 50 mM [Na⁺], 5.7 mM [Mg²⁺], the standard enthalpy and entropy change for the hybridization reactions between (a) pseudo-target strand and the single-stranded oligonucleotide (ΔH°=−238.90 kcal/mole, ΔS°=−660.72 e.u.) and (b) target strand and the single-stranded oligonucleotide (ΔH°=−222.40 kcal/mole, ΔS°=−618.90 e.u.) was calculated. Using the above equations, the fold-amplification after 35 cycles versus the annealing temperature for both pseudo-target (FIG. 8A, left, thin dashed line) and target template (FIG. 12A, thin solid line) was plotted. The ratio between ‘fold-amplification after 35 cycles for target template’ and ‘fold-amplification after 35 cycles for pseudo-target template’ for different annealing temperatures was also calculated (FIG. 8A, left, thick solid line). This ratio is defined as ‘selectivity of amplification.’ It is clear that significant discrimination (selectivity of ˜10⁵) is achieved only when the annealing temperature is near the melting temperature (Tₘ) of the template:oligo duplex. When the annealing temperature is below this Tₘ, both pseudo-target and target templates are blocked, and when the annealing temperature is above this Tₘ, neither pseudo-target nor target template is blocked. Either case results in poor selectivity. One consequence of such behavior of the single-stranded oligonucleotide blocker is that the blocker cannot to be very long (e.g., it cannot be longer than 15 to 25 bases, depending on the chemical nature and sequence of the blocker). For example, a Tₘ of greater than about 80° C. would not result in efficient primer binding and polymerase extension. Thus, the “scope” of target/pseudo-target sequence is limited with wild type-blocking PCR.

dsBlocker Amplification.

The presence of the balancing toehold domains and the protector strand permits ultra-specific hybridization between the pseudo-target template and the blocker strand at temperature substantially lower than the Tₘ of the template:blocker hybridization (Zhang, D. Y., et al. Nat Chem, 4(3):208-214, 2012). Thus, the blocker strand of a dsBlocker, in some embodiments, may be designed to be longer than the single-stranded oligonucleotide primer of a traditional wild type-blocking PCR. Further, the dsBlockers of the present disclosure, in some embodiments, permit high selectivity of nucleic acid amplification across a wide range of temperatures. However, this phenomenon was only tested at temperatures below 37° C. which are not suitable for PCR. Moreover, it was not obvious how the sequence specificity in one binding step translates to the selectivity of an exponential amplification. To estimate the performance of dsBlocker amplification, a dsBlocker of the present disclosure was designed to have the following sequence: 5′-GCTTAGATA[T]CGIGCACAAATAG CGTCGGAT(GGGCG)tcttcttca-3′ (SEQ ID NO:24), where the balancing toehold (BT) domain is shown in lower case, and the initial toehold (IT) domain and the branch migration (BM) domain are shown in upper case on the left and right side of the symbol ‘I’, respectively. The base that differentiates the pseudo-target and the target template is enclosed by brackets. The sequence in the parentheses was not present in the single-stranded oligonucleotide primer, described above, but is nevertheless derived from the target sequence and is part of the branch migration domain. Thus, mutations in this region of the target can be identified by the dsBlocker of the present disclosure but not the traditional single-stranded oligonucleotide primer (i.e., the dsBlocker has a “broader scope” of target sequence).

The protector strand of the dsBlocker was designed to have the following sequence: 5′-tgaagaaga(CGCCC)ATCCGACGCTATTTGTGC-3′ (SEQ ID NO:25), where the balancing toehold domain and the branch migration domain are shown in lower and upper cases, respectively. The sequence in parentheses is complementary to the sequence in parentheses of the blocker strand. The concentrations of the blocker strand and the protector strand were set to be 100 nM and 150 nM, respectively. Using the above equations, the fold-amplification after 35 rounds for the pseudo-target (FIG. 8, right, thin dashed line) and target (FIG. 8, right, thin solid line) template at different annealing temperature was calculated. The selectivity of amplification versus annealing temperature (FIG. 8, right, thick solid line) was also plotted. It is clear from this analysis that optimal selectivity can be achieved with the dsBlocker of the present disclosure under a surprisingly wide range of annealing temperatures that are suitable for PCR, due to the effect of entropy cancellation.

### Methods

Some aspects of the present disclosure provide methods that comprise combining in a reaction mixture a target nucleic acid and a primer that comprises a sensing module and a priming module, wherein the sensing module binds to a target nucleic acid and, when bound to the target nucleic acid, recruits and activates the priming module, wherein the activated priming module binds to the target nucleic acid upstream of the sensing module.

The methods may further comprise incubating the reaction mixture under conditions that result in recruitment of the primer module to the sensing module, activation of the priming module, and binding of the minimal primer sequence to the target nucleic acid. In some embodiments, the methods further comprise incubating the reaction mixture under conditions that result in amplification of the target nucleic acid.

Retro-activated (RA) primers of the present disclosure, in some embodiments may be used in nucleic acid synthesis reactions, including amplification reactions. In some embodiments, the temperature of the reaction solutions may be sequentially cycled between a denaturing state, an annealing state, and an extension state for a predetermined number of cycles. The actual times and temperatures can depend on the enzyme, primer, and target nucleic acid of interest.

For any given reaction, denaturing states may range from about 75° C. to about 100° C. The annealing temperature and time can influence the specificity and efficiency of the primer and other molecules binding to a particular target nucleic acid and may be important for particular synthesis reactions.

For any given reaction, annealing states may range from about 20° C. to about 75° C., or about 20° C. to about 85° C. In some embodiments, the annealing state may be performed at about 20° C. to about 25° C., about 25° C. to about 30° C., about 30° C. to about 35° C., or about 35° C. to about 40° C., about 40° C. to about 45° C., about 45° C. to about 50° C., about 50° C. to about 55° C., about 55° C. to about 60° C., about 60° C. to about 65° C., about 65° C. to about 70° C., about 70° C. to about 75° C., about 75° C. to about 80° C., about 80° C. to about 85° C. In some embodiments, the annealing state may be performed at room temperature (e.g., 20° C. or 25° C.). In some embodiments, the annealing state may be performed at a temperature of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C. or 50° C.

Extension temperature and time may impact the product yield and are understood to be an inherent property of polymerase used. For a given polymerase, extension states may range from about 60° C. to about 75° C. It is to be understood that the polymerase may be able to extend the primer in states other than the extension state of PCR.

In some embodiments, the polymerase used may have strand displacement activity. Examples include Vent polymerase, Bsm polymerase, Bst polymerase, Csa polymerase and 96-7 polymerase. In some embodiments, the polymerase used is characterized simply by its ability to catalyzes polymerization of nucleotides into a nucleic acid strand, including thermostable polymerases and reverse transcriptases (RTases). Examples include Bacillus stearothermophilus pol I, Thermus aquaticus (Taq) pol I, Pyrococcus furiosus (Pfu), Pyrococcus woesei (Pwo), Thermus flavus (Tfl), Thermus thermophilus (Tth), Thermus litoris (Tli) and Thermotoga maritime (Tma). These enzymes, modified versions of these enzymes, and combination of enzymes, are commercially available from vendors including Roche, Invitrogen, Qiagen, Stratagene, and Applied Biosystems. Representative enzymes include PHUSION® (New England Biolabs, Ipswich, Mass.), Hot MasterTaq™ (Eppendorf), PHUSION® Mpx (Finnzymes), PyroStart® (Fermentas), KOD (EMD Biosciences), Z-Taq (TAKARA), and CS3AC/LA (KlenTaq, University City, Mo.).

Salts and buffers include those familiar to those skilled in the art, including those comprising MgCl₂, and Tris-HCl and KCl, respectively. Buffers may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP), and are also added to a reaction adequate amount for amplification of the target nucleic acid.

### Two-Step Selective Amplification Method

Some embodiments provide a two-step (library-construction) method for selective amplification of DNA. The method, in some embodiments, combines dsBlockers, RA primers and single-molecule barcoding (Kinde I et al. PNAS 108(23): 9530-35, incorporated by reference herein) (FIG. 8C). In some embodiments, the method includes an “adaptor tagging” step and a “mutation enrichment” step. In the adaptor tagging step, multiple (e.g., 10, 15, 20, 25, 30, 35, 40, 45, 50, 55, 60, 65, 70, 75, 80, 85, 90, 95, 100, 105, 110, 115, 120, 125, 130, or more) different genome loci (e.g., each 40-60 bp, or 30-50 bp long) undergo various (e.g., 2, 3, 4, 5, 6, 7, 8, 9 or 10) cycles of PCR in the presence a large set of primers that append a single-molecule barcode, sample index and sequencing primers (collectively referred to as “adaptors”) to the target sequence. In the mutation enrichment stage, a dsBlocker primer and a RA primer may be used in combination to selectively amplify target DNA that, for example, harbors a mutation (FIG. 8C).

As shown in FIG. 8C, the blocker strand (also referred to as pro-clamp strand) competes with the protector strand (also referred to as the anchor strand) of a forward RA primer, where the outcome of the competition typically depends on the sequence of the target DNA. The blocker strand competes favorably on the wild-type template, but unfavorably on the mutant templates, resulting in selective amplification of the mutants, for example. The product may be diluted, quantified, pooled and subject to MiSeq sequencing to acquire (only) approximately 1,000 reads per locus, in some embodiments.

In some embodiments, the method does not contain a purification step, thus, the method, which may also include a sequencing step, can be completed within a day (e.g., one day, less than a day, 12 hours or less, or 6 hours or less), for example.

### Applications

The present disclosure may be used, in some embodiments, to detect circulating tumor DNA (ctDNA), which is released from tumor cells (e.g., into circulating blood). Circulating tumor DNA may be distinguished from DNA of the same locus, which is released from normal cells, by the presence of tumor-specific mutations.

RA primers may be used, in some embodiments, to detect DNA released from donor cells in an organ transplant recipient. Monitoring organ rejection after transplantation requires the detecting and quantification of DNA released from donor cells in an excess background of DNA released from recipient cells. Thus, in some embodiments, the present disclosure provides methods of monitoring organ rejection in a recipient subject after organ transplantation from a donor subject, the methods comprising contacting a sample obtained from the recipient subject with an RA primer designed to bind (e.g., via Domains 3A and 4 of the pro-anchor strand and via Domain 6A of the pro-primer strand) to a target donor allele.

RA primers may be used, in some embodiments, to detect the presence of drug-resistant microorganisms in a sample, which requires the detection and quantification of nucleic acids from drug-resistant microorganisms in an background of nucleic acids from drug-sensitive counterparts. Thus, in some embodiments, the present disclosure provides methods of detecting nucleic acids from drug-resistant microorganisms, the methods comprising contacting a sample obtained from the recipient subject with an RA primer designed to bind (e.g., via Domains 3A and 4 of the pro-anchor strand and via Domain 6A of the pro-primer strand) to a target nucleic acid from a drug-resistant microorganism.

In some embodiments, the sample is a tissue sample or a biological fluid sample such as, for example, a blood (e.g., plasma or serum) sample, saliva sample, or a urine sample. Other biological samples may be used in accordance with the invention and are described elsewhere herein.

In some embodiments, the microorganisms are bacterial cells such as, for example, Escherichia coli cells.

### Compositions and Kits

Some aspects of the present disclosure comprise compositions and/or kits that include RA primers, as provided herein.

In some embodiments, a composition and/or a kit may comprise a polymerase, as provided herein. For example, a composition and/or kit may comprise Vent polymerase, Bsm polymerase, Bst polymerase, Csa polymerase, 96-7 polymerase, Bacillus stearothermophilus pol I, Thermus aquaticus (Taq) pol I, Pyrococcus furiosus (Pfu), Pyrococcus woesei (Pwo), Thermus flavus (Tfl), Thermus thermophilus (Tth), Thermus litoris (Tli) or Thermotoga maritime (Tma).

In some embodiments, a composition and/or a kit may comprise a salt and/or buffer, including those comprising MgCl₂, and Tris-HCl and KCl, respectively. Buffers may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP).

The present disclosure is further described by the following numbered paragraphs:

1. A primer, comprising a sensing module and a priming module, wherein the sensing module binds to a target nucleic acid and, when bound to the target nucleic acid, recruits and activates the priming module, wherein the activated priming module binds to the target nucleic acid upstream of the sensing module.
2. The primer of paragraph 1, wherein the sensing module comprises a partially double-stranded nucleic acid comprising a first nucleic acid strand bound to a second nucleic acid strand, wherein the first strand comprises

(a) a 5′ domain that includes sequence complementary to the priming module,

wherein a portion of the sequence of (a) is complementary to and bound to the second strand, thereby forming a double-stranded region, and

(b) a 3′ domain that includes sequence complementary to the target nucleic acid,

wherein a portion of the sequence of (b) is complementary to and bound to the second strand, thereby forming a double-stranded region.

3. The primer of paragraph 1 or 2, wherein the priming module comprises a partially double-stranded nucleic acid comprising a first nucleic acid strand bound to a second nucleic acid strand, wherein the first strand comprises

(c) a 5′ domain that includes sequence complementary to the sensing module,

wherein a portion of the sequence of (c) is complementary to and bound to the second strand, thereby forming a double-stranded region, and

(d) a 3′ domain that includes a chemical linker attached to a minimal primer sequence,

wherein the minimal primer sequence is complementary to the target nucleic acid and is bound to the second strand, thereby forming a double-stranded region.

4. The primer of paragraph 1 or 2, wherein the priming module comprises a partially double-stranded nucleic acid comprising a first nucleic acid strand bound to a second nucleic acid strand, wherein the first strand comprises

(c) a 5′ domain that includes sequence complementary to the sensing module, wherein a portion of the sequence of (c) is complementary to and bound to the second strand, thereby forming a double-stranded region, and

(d) a 3′ domain attached to a minimal primer sequence, wherein the minimal primer sequence is complementary to the target nucleic acid and is bound to the second strand, thereby forming a double-stranded region.

5. The primer of paragraph 4, wherein the 3′ domain includes a chemical linker attached to the minimal primer.
6. The primer of paragraph 4, wherein the 3′ domain includes a polymerase-stopping or polymerase-pausing moiety attached to the minimal primer.
7. The primer of paragraph 1, wherein the sensing module comprises:

(a) a first nucleic acid strand containing, in a 5′ to 3′ direction, Domain 1A, Domain 2A, Domain 3A and Domain 4A, wherein Domain 1A and Domain 4A are unbound, and wherein Domain 3A and Domain 4A are complementary to the target nucleic acid, and

(b) a second nucleic acid strand containing, in a 5′ to 3′ direction, Domain 3B and Domain 2B, wherein Domain 3B and Domain 2B are respectively complementary to and bound to Domain 3A and Domain 2A of the first strand of (a).

8. The primer of paragraph 7, wherein the priming module comprises:

(c) a first nucleic acid strand containing, in a 5′ to 3′ direction, Domain 2B, Domain 1B, Domain 5A, a linker molecule, and Domain 6A, wherein Domain 2B and Domain 1B are respectively complementary to Domain 2A and Domain 1A of the first strand of (a), and Domain 2B is unbound, and

(d) a second nucleic acid containing, in a 5′ to 3′ direction, Domain 6B, Domain 7, Domain 5B and Domain 1A, wherein Domain 6B, Domain 5B and Domain 1A are respectively complementary to and bound to Domain 6A, Domain 5A and Domain 1B of the first strand of (c), and wherein Domain 7 is optionally unbound.

9. The primer of any one of paragraphs 1-8, wherein the sensing module is linked to the priming module via a linker molecule.
10. The primer of paragraph 9, wherein the linker molecule is a chemical linker.
11. The primer of paragraph 9, wherein the linker molecule is a single-stranded nucleic acid.
12. A nucleic acid molecule, comprising:

(a) a first nucleic acid strand;

(b) a second nucleic acid strand comprising
(i) a 3′ domain that includes sequence complementary to and bound to the first strand, thereby forming a first double-stranded domain, and
(ii) a 5′ domain that includes sequence complementary to and bound to a third nucleic acid strand, thereby forming a second double-stranded domain; and

(c) the third nucleic acid strand comprising
(i) a 5′ domain that contributes to the second double-stranded domain of (b)(ii), and
(ii) a 3′ domain that includes a chemical linker attached to a minimal primer sequence, wherein the minimal primer sequence is complementary to the target nucleic acid.
   13. The nucleic acid molecule of paragraph 12, wherein the minimal primer sequence is bound to the first strand, upstream from the first double-stranded region.
   14. A method comprising combining in a reaction mixture a target nucleic acid and the primer of any one of paragraphs 1-11.
   15. A method comprising combining in a reaction mixture a target nucleic acid and a primer that comprises a sensing module and a priming module, wherein the sensing module binds to a target nucleic acid and, when bound to the target nucleic acid, recruits and activates the priming module, wherein the activated priming module binds to the target nucleic acid upstream of the sensing module.
   16. A method, comprising combining in a reaction mixture a target nucleic acid with a primer that comprises a sensing module and a priming module, wherein

the sensing module comprises a partially double-stranded nucleic acid comprising a first nucleic acid strand bound to a second nucleic acid strand, wherein the first strand comprises
(a) a 5′ domain that includes sequence complementary to the priming module, wherein a portion of the sequence of (a) is complementary to and bound to the second strand, and
(b) a 3′ domain that includes sequence complementary to the target nucleic acid,
wherein a portion of the sequence of (b) is complementary to and bound to the second strand, and

the priming module comprises a partially double-stranded nucleic acid comprising a first nucleic acid strand bound to a second nucleic acid strand, wherein the first strand comprises

(c) a 5′ domain that includes sequence complementary to the sensing module,

wherein a portion of the sequence of (c) is complementary to and bound to the second strand, and

(d) a 3′ domain that includes a chemical linker attached to a minimal primer sequence, wherein the minimal primer sequence is complementary to the target nucleic acid and is bound to the second strand.

17. The method of paragraph 16 further comprising incubating the reaction mixture under conditions that result in recruitment of the primer module to the sensing module, activation of the priming module, and binding of the minimal primer sequence to the target nucleic acid.
18. The method of paragraph 17 further comprising incubating the reaction mixture under conditions that result in amplification of the target nucleic acid.
19. A composition comprising the primer of any one of paragraphs 1-11, or the nucleic acid molecule of paragraph 12 or 13.
20. The composition of paragraph 19 further comprising the target nucleic acid.
21. A kit comprising the primer of any one of paragraphs 1-11.
22. A kit comprising at least two of the primer of any one of paragraphs 1-11, wherein each primer is designed to bind to a different target nucleic acid.
23. The kit of paragraph 21 or 22 further comprising at least one of the following reagents: buffer, deoxyribonucleotide triphosphates (dNTPs), nuclease-free water and polymerase.
24. The method of paragraph 18, wherein the conditions that result in amplification of the target nucleic acid include incubating the reaction mixture at a temperature of 50° C.-70° C. for a time sufficient to results in amplification of the target nucleic acid.

### EXAMPLES

### Example 1

To test the effectiveness of the RA primer design strategy shown in FIGS. 4A-5, a pair of similar primers was created. Table 1 lists the RA primer sequences.

TABLE 1
RA Primer Sequences

| | Sequence | Comments |
|---|---|---|
| Forward Primer | | |
| Pro-Anchor strand | GCAATCGTCGccctactatcctcctc | /3InvdT/indicates |
| (fPA) | GTTCAAACTGATGGGACCC | an inverted dT at the |
| | ACTCCA/3InvdT/ | 3′ end to prevent its |
| | (SEQ ID NO: 1) | extension |
| Anti-Anchor | ATCAGTTTGAAC gaggaggatagtag | /3InvdT/indicates |
| strand (fAA) | /3InvdT/ | an inverted dT at the |
| | (SEQ ID NO: 2) | 3′ end to prevent its |
| | | extension |
| Pro-Primer strand | gaggaggatagtaggg | /iSp18/is an 18- |
| (fPP) | CGACGATTGCATCTAGTCC | atom, hexaethylene |
| | /iSp18//iSp18/CTCAGAGTTGCAG | glycol linker |
| | (SEQ ID NO: 3) | |
| Anti-Primer strand | CTGCAACTCTGAG TAGAT | /3InvdT/indicates |
| (fAP) | GCAATCGTCG/3InvdT/ | an inverted dT at the |
| | (SEQ ID NO: 4) | 3′ end to prevent its |
| | | extension |
| Reverse Primer | | |
| Pro-Anchor (rPA) | TGATCCGATGACagggcaaatacgaga | /3InvdT/indicates |
| | TACTTACTACACCT CAGATA | an inverted dT at the |
| | TATTTCTTCA TGAAGAC | 3′ end to prevent its |
| | /3InvdT/ | extension |
| | (SEQ ID NO: 5) | |
| Anti-Anchor (rAA) | AGGTGTAGTAAGTA tctcgtatttgc | /3InvdT/indicates |
| | /3InvdT/ | an inverted dT at the |
| | (SEQ ID NO: 6) | 3′ end to prevent its |
| | | extension |
| Pro-Primer (rPP) | tctcgtatttgccct GTCATCGGATCA | /iSp18/is an 18- |
| | AGCTAGT/iSp18//iSp18/ | atom, hexaethylene |
| | TCCATGGTGCAAG | glycol linker |
| | (SEQ ID NO: 7) | |
| Anti-Primer (rAA) | CTTGCACCATGG tt | /3InvdT/indicates |
| | AGCTTGATCCGATGAC a | an inverted dT at the |
| | /3InvdT/ | 3′ end to prevent its |
| | (SEQ ID NO: 8) | extension |

As a comparison, a pair of primers based on those described in International Pub. No. WO/2015/010020 was also created. The primers are referred to as “foresight primers,” each having a specificity domain-containing strand (the SD strand), a priming domain-containing strand (the PD strand), and a competitive domain-containing strand (the CD strand). Table 2 lists the foresight primer sequences.

TABLE 2
Foresight Primer Sequences

| | Sequence | Comments |
|---|---|---|
| Forward Primer | | |
| SD strand | GGACTAGATATCCATGCAATCGTCG | /3InvdT/indicates |
| (fSD) | ccctactatcctcctcGTTCAAA | an inverted dT at the |
| | CTGATGGGACCCACTCCA/3InvdT/ | 3′ end to prevent its |
| | (SEQ ID NO: 9) | extension |
| PD strand | CGACGATTGCATGGATATCTAGTCC | /iSp18/is an 18- |
| (fPD) | /iSp18//iSp18/ | atom, hexaethylene |
| | CTCAGAGTTGCAG | glycol linker |
| | (SEQ ID NO: 10) | |
| CD strand | ATCAGTTTGAAC gaggaggatagtag | /3InvdT/indicates |
| (fCD) | /3InvdT/ | an inverted dT at the |
| | (SEQ ID NO: 11) | 3′ end to prevent its |

| | | |
|---|---|---|
| | | extension |
| Reverse Primer | | |
| SD strand | ACTAGCTGACAGTTGATCCGATGAC | /3InvdT/indicates |
| (rSD) | agggcaaatacgaga TACTTACTACACCT | an inverted dT at the |
| | CAGATATATTTCTTCATGAAG | 3′ end to prevent its |
| | /3InvdT/ | extension |
| | (SEQ ID NO: 12) | |
| PD strand | GTCATCGGATCAACTGTCAGCTAGT | /iSp18/is an 18- |
| (rPD) | /iSp18//iSp18/TCCATGGTGCAAG | atom, hexaethylene |
| | (SEQ ID NO: 13) | glycol linker |
| CD strand | GGTGTAGTAAGTAtctcgtatttgc/ | /3InvdT/indicates |
| (rCD) | 3InvdT/ | an inverted dT at the |
| | (SEQ ID NO: 14) | 3′ end to prevent its |
| | | extension |

Two double-stranded DNAs (synthesized as gBlocks™) were used as templates (Integrated DNA Technologies (IDT, Coralville, Iowa)). The first template (TempA,) has the following sequence: CTCAGAGTTGCAGATATCCGGTCGCCTAAG ccagacaactgttcaaactgatgggacccactccatcgagatttctctgtagctagaccaaaatcacctatttttactgtgaggtcttcatg aa gaaatatatctgaggtgtagtaagtaaaggaaaacaGACGGCTCGACTGATATCTTGC ACCATGGA (SEQ ID NO: 15). The second template (TempB) has the following sequence: CTCAGAGTTGCAGATATCCGGTCGCCTAAGtgacaaagaacagctca aagcaatttcta cacgagatcctctctctgaaatcactgagcaggagaaagattttctatggagtcacaggtaag tgctaaaatggagattctcGACG GCTCGACTGATATCTTGCACCATGGA (SEQ ID NO: 16). The templates share the same flanking sequences (capitalized), on which the minimal primers of the RA primers and priming domains of the foresight primers bind. However, the sensing modules of the RA Primers and the specificity domains of the foresight primers recognize only TempA, not TempB. Therefore, only TempA is amplified.

Two quantitative PCR reactions using the foresight primers were performed. The first reaction contained 1× Standard Taq Buffer (NEB, Ipswich, Mass.), 3 mM MgSO₄, 1 U of Pfu(exo-) (Agilent, Lexington, Mass.), 0.2 mM of each of the 4 dNTPs, 0.01 mM of SYTO-9, 100 Nm fSD, 120 nM of fPD, 200 nM of fCD, 100 nM rSD, 120 nM of rPD, 200 nM of rCD, and 0.85 fM of TempA in a 20-microliter volume. The second reaction was identical to the first reaction except TempB was used in place of TempA. The quantitative PCR program was the following: (1) 95° C., 1 min; (2) 95° C., 15 s; (3) 60° C., 1 min 30 s; (4) 50° C., 30 s; (5) 70° C., 30 s; (6) 80° C., 30 s [Read]; (7) Goto (2) for 59 additional cycles. The amplification kinetics traces of the first and second reactions are shown by the solid and dashed lines in FIG. 6B, respectively. Although TempA (the intended template) was amplified efficiently, TempB (the unintended template) was also amplified with a delay of ˜18 cycles.

The rPD was then shortened by one nucleotide at its 3′ end to form rPD.2, having the following sequence: GTCATCGGATCAACTGTCAGCTAGT/iSp18//iSp18/TCCATGGTGCAA (SEQ ID NO: 17). Two additional quantitative PCR reactions were then performed, referred to as the third reaction and the fourth reaction. The third reaction and the fourth reaction were identical to the first reaction and second reaction, respectively, with the exception that in both reactions, rPD.2 was used in place of rPD. Graphs representative of the amplification kinetics are shown in FIG. 6B—the third reaction and fourth reaction are depicted as a solid line and a dashed line, respectively. Amplification in both reactions was reduced, although amplification of TempB (the unintended template) was still observed.

Two additional quantitative PCR reactions were then performed. RA primers were used in place of foresight primers. The fifth reaction contained 1× Standard Taq Buffer (NEB, Ipswich, Mass.), 3 mM MgSO₄, 1 U of Pfu(exo-) (Agilent, Lexington, Mass.), 0.2 mM of each of the 4 dNTPs, 0.01 mM of SYTO-9, 100 nM fPA, 200 nM of fAA, 120 nM of fPP, 240 nM of fAP, 100 nM rPA, 200 nM of rAA, 120 nM of rPP, 240 nM of rAP, and 0.85 fM of TempA in 20-microliter volume. The sixth reaction was identical to the fifth reaction, with the exception that TempB was used in place of TempA. Graphs representative of the amplification kinetics are shown in FIG. 6C—the fifth reaction and sixth reaction are depicted as a solid line and a dashed line, respectively. Unexpectedly, while TempA was amplified with kinetics similar to the third reaction (FIG. 6B, solid line), the amplification of TempB (the unintended template) was completely absent. Thus, the RA primers are superior to the primers of the prior art (compare FIG. 6C and FIG. 6B).

### Example 2

An RA primer can be used in combination with the “dsBlocker” described in International Pub. No. WO/2015/010020 to selectively amplify mutant sequences while suppressing the amplification of the wild-type sequence. To achieve this, a dsBlocker and an RA primer are engineered such that the blocker strand of the dsBlocker and the pro-anchor strand of the RA primer do bind simultaneously to the target nucleic acid. One way to achieve this is to design Domain 4A of the pro-anchor domain of the RA primer and the single-stranded region of the dsBlocker to share the same binding site on the template. One such example, showing an RA primer and a dsBlocker used in combination to achieve selective amplification of a mutant DNA, follows.

As described above, a “dsBlocker” refers to the following: a thermodynamic, partially double-stranded nucleic acid with enhanced target specificity having first and second nucleic acid strands arranged into (a) one double-stranded pseudo-target non-specific domain, (b) one double-stranded pseudo-target specific domain, and (c) one single-stranded pseudo-target specific domain contributed to by the first nucleic acid strand, wherein the double-stranded pseudo-target non-specific domain has a standard free energy approximately equal to the standard free energy for the single-stranded pseudo-target specific domain bound to a pseudo-target nucleic acid, and wherein the 3′ end of the first nucleic acid strand and the 3′ of the second nucleic acid strand are non-extendable. Nucleic acids to which a dsBlocker binds may be characterizes as a “target” nucleic acid or a “pseudo-target” nucleic acid. A pseudo-target nucleic acid (e.g., a wild-type allele) is typically in abundance relative to a target nucleic, which typically refers to a mutated, rare allele of interest. For example, a target and a pseudo-target may differ by only a single nucleotide (or nucleotide base pair, in the form of mutation, insertion, or deletion).

The synthetic double-stranded DNA template (TempA2) used in this example has the following sequence: CTCAGAGTTGCAGATATCCGGTCGCCTAAGccagacaactg ttcaaactgatgggacccactccatcgagatttc[A]ctgtagctagaccaaaatcacctatttttactgtgaggtcttcatgaagaaatat atctgaggtgtagtaagtaaaggaaaacaGACGGCTCGACTGATATCTTGCACCATGGA (SEQ ID NO: 18). TempA2 differs from TempA of Example 1 by one nucleotide, i.e., the base ‘A’ in brackets in TempA2 replaces the base ‘T’ in TempA. A dsBlocker was designed to contain (1) the following ‘blocker strand’ sequence: GGGACCCACTCCA TCGAGATTTCACTGTAGCTAGACCAAAATCcaagcgacgagaa mAmA/3InvdT/(SEQ ID NO: 19), where “mA” denotes 2′-OMe-A; and (2) the following ‘protector strand’ ctcgtcgcttgGATTTTGGTCTAGCTACAGTGAAATCTCGA mAmA/3InvdT/(SEQ ID NO: 20). The dsBlocker was designed to bind TempA2 with an affinity higher than TempA.

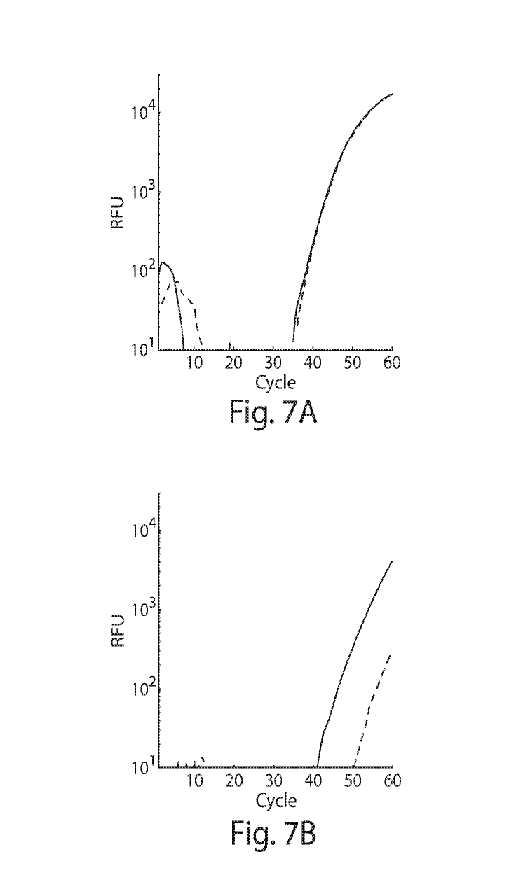

Using a quantitative polymerase chain reaction (qPCR) assay, similar to the assays described in Example 1, it was confirmed that TempA2 and TempA can be amplified using the RA primer with practically indistinguishable efficiency (FIG. 7A). In contrast, when 150 nM of the ‘blocker strand’ and 250 nM of the ‘protector strand’ were added to the reaction, TempA2 is amplified with a much lower efficiency than TempA, indicating that the amplification of TempA2 is suppressed by the dsBlocker.

### Example 3

In this Example, a library-construction platform was developed (FIG. 8C), which combines various single-molecule detection assays (WO/2015/010020, which is incorporated herein by reference; FIG. 8A) with the RA primers of the present disclosure (FIG. 8B) to provide, inter alia, greater than 100-fold target enrichment, multiplexing capability (e.g., enrichment of approximately 100 genomic loci in less than 10 reactions, compatibility with low-input DNA (e.g., approximately 5 ng or less), efficient and “hands-off automation,” the ability to report the copy number of mutant DNA, the capability to process at least 12-24 samples at a time, and low cost (e.g., reagent and sequencing costs of less than $100/sample).

The platform, as provided herein and depicted in FIG. 8C, has at least two stages: adaptor tagging and mutation enrichment (FIG. 8C, panel (a), top). In the adaptor tagging stage, for example, up to 100 genome loci (each 30- to 50-bp long) undergo, for example, 4 cycles of PCR in the presence a large set of primers that append single-molecule barcode (also referred to as unique molecule identifier, or UMI), sample index and sequencing primers (collectively called ‘adaptors’) to the target sequence. In the mutation enrichment stage (FIG. 8C, panel (a), bottom), the RA primers and the primers described in International Pub. No. WO/2015/010020 (and shown in FIG. 8A) are used in combination to selectively amplify target DNAs that harbor mutations.

Since most of the reactions involved in this method are entropy-neutral strand-displacement reactions, this platform is referred to as “Isoentropic Wildtype Suppressive Enrichment PCR,” or “iWISE-PCR.” As shown in FIG. 8C, a key principle of the iWISE-PCR is that the Pro-Clamp strand competes with the Anchor of the forward RA Primer (also referred to in the figure as a forward ifPRimer), where the outcome of the competition depends on the sequence of the target DNA: Pro-Clamp competes favorably on the wild-type template, but unfavorably on the mutant templates, resulting in selective amplification of the mutants. The product of protocol is diluted, quantified, pooled and subject to MiSeq sequencing to acquire (only) approximately 1,000 reads per locus. The method is therefore referred to as iWISE-Seq. In some embodiments, library construction contains no purification step, therefore the entire procedure including sequencing can be completed within a day.

### Example 4

A qPCR assay was designed to assess the performance of iWISE-PCR, where pure, synthetic, adaptor-containing wild-type DNA or mutant DNA were used as the template and product formation was monitored by a SYBR Gold-like dye (see Example 1). Using this assay, the un-optimized iWISE-PCR shown in Example 2can suppress the amplification of wild-type sequence by 10 cycles relative to the mutant sequence (approximately 100-fold enrichment of mutant). More (e.g., 10) different model sequence are used to perform a study, where the binding energy (ΔG, which is in turn determined by sequence in a predictable fashion) of each domain is combinatorially varied in the practical range, and the amplification efficiency for each template is measured using a dilution series with this qPCR assay. The amplification efficiency of the wild-type and mutant template is, for example, <1.3 and >1.8, respectively. This criterion is chosen because such difference in amplification efficiency can be safely achieved for most mutations based on thermodynamic analyzes, and result in 17,000-fold enrichment of mutant after 30 cycles of PCR [i.e., (1.811.3)³⁰≈17,400]. In practice, the enrichment factor is determined by the fidelity of DNA polymerase. Based on this model, common proofreading polymerases (e.g., Pfu, PHUSION®, KAPA HIFI™) have sufficient fidelity to yield>1,000-fold enrichment for all types of base changes. This ‘1.8/1.3’ criterion is met with 16 mutants representing all 12 potential types of base changes for 10 different (all) wild-type model sequences.

An NGS-based assay is used to test the limit of multiplexing. The 10 different wild-type model sequence are mixed and a representative mutant for each model sequence is spiked in at the abundance of 0.1% each. Different multiplexed enrichment reactions are carried out with the multiplexity (number of enrichment reactions in one tube) varying from 1 to 10. In these reactions, the total concentration of oligonucleotide may remain constant, which means the concentration of each reagent decreases as the multiplexity increases. Accordingly, the annealing time of iWISE-PCR is extended to ensure completion of priming. The iWISE-PCR product is subject to MiSeq sequencing to acquire 1,000× multiplexity total reads after Q30-based filtering (the reads are randomly down-sampled if the actual number of reads is larger). The highest multiplexity achievable is defined as the multiplexity at which: (1) iWISE-PCR product is visible on BioAnalyzer; (2) more than 99.9% of total reads are on target; (3) at least 500 reads are recorded for each target sequence; (4) >30% of the total reads is from the spiked-in mutant; (5) no false-positive mutation sequence represents >1% of total reads.

### Example 5

In this Example, the feasibility of the entire iWISESeq protocol is demonstrated. iWISE-PCR is performed using template that is the product of the Adaptor Tagging stage. A panel of 20 loci are tested. The panel includes hotspots in exons 18, 19, 20, 21 of EGFR, exons 1 and 2 of KRAS and NRAS, exons 9 and 20 of PIK3CA, exon 15 of BRAF, and 9 more regions in TP53 and PIK3CA. This panel represents the most common driver mutations in NSCLC and can be used in companion diagnostics of this disease to guide the usage of targeted compounds such as Tarceva and Xalkori, and in clinical trials of investigational TKIs such as AZD9291 and CO1686. Thus, this panel is termed ‘NSCLC CDx’ panel.

The entire two-stage platform described in Example 4 as well as MiSeq sequencing is performed with 5 ng of sheared genomic DNA from healthy donors (commercially available) with synthetic mutant DNA spiked in at abundance of 0.02% to 1%. A total of 20,000 Q30-filtered reads are obtained from the sequencing run (random down-sampling may be carried out). The data meets the following criteria: (1) when the product of the iWISE-PCR is analyzed using electrophoresis (e.g., BioAnalyzer), no erroneous band is observed; (2) off-target reads represent <1% of the final sequencing library; (3) each loci records at least 500 reads; (4) 0.01% of mutants is accurately detected and quantified; and (5) no false-positive mutation is observed.

Next, commercially available reference materials (from Horizon Dx) are used to fully characterize the analytical sensitivity and specificity of the NSCLC CDx panel.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A primer, comprising a sensing module and a priming module, wherein the sensing module binds to a target nucleic acid and, when bound to the target nucleic acid, recruits and activates the priming module, wherein the activated priming module binds to the target nucleic acid upstream of the sensing module.

2. The primer of claim 1, wherein the sensing module comprises a partially double-stranded nucleic acid comprising a first nucleic acid strand bound to a second nucleic acid strand, wherein the first strand comprises
(a) a 5′ domain that includes sequence complementary to the priming module,
wherein a portion of the sequence of (a) is complementary to and bound to the second strand, thereby forming a double-stranded region, and
(b) a 3′ domain that includes sequence complementary to the target nucleic acid,
wherein a portion of the sequence of (b) is complementary to and bound to the second strand, thereby forming a double-stranded region.

3. The primer of claim 1, wherein the priming module comprises a partially double-stranded nucleic acid comprising a first nucleic acid strand bound to a second nucleic acid strand, wherein the first strand comprises
(c) a 5′ domain that includes sequence complementary to the sensing module,
wherein a portion of the sequence of (c) is complementary to and bound to the second strand, thereby forming a double-stranded region, and
(d) a 3′ domain that includes a chemical linker attached to a minimal primer sequence, wherein the minimal primer sequence is complementary to the target nucleic acid and is bound to the second strand, thereby forming a double-stranded region.

4. The primer of claim 1, wherein the priming module comprises a partially double-stranded nucleic acid comprising a first nucleic acid strand bound to a second nucleic acid strand, wherein the first strand comprises
(c) a 5′ domain that includes sequence complementary to the sensing module, wherein a portion of the sequence of (c) is complementary to and bound to the second strand, thereby forming a double-stranded region, and
(d) a 3′ domain attached to a minimal primer sequence, wherein the minimal primer sequence is complementary to the target nucleic acid and is bound to the second strand, thereby forming a double-stranded region.

5. The primer of claim 4, wherein the 3′ domain includes a chemical linker attached to the minimal primer.

6. The primer of claim 4, wherein the 3′ domain includes a polymerase-stopping or polymerase-pausing moiety attached to the minimal primer.

7. The primer of claim 1, wherein the sensing module comprises:
(a) a first nucleic acid strand containing, in a 5′ to 3′ direction, Domain 1A, Domain 2A, Domain 3A and Domain 4A, wherein Domain 1A and Domain 4A are unbound, and wherein Domain 3A and Domain 4A are complementary to the target nucleic acid, and
(b) a second nucleic acid strand containing, in a 5′ to 3′ direction, Domain 3B and Domain 2B, wherein Domain 3B and Domain 2B are respectively complementary to and bound to Domain 3A and Domain 2A of the first strand of (a).

8. The primer of claim 7, wherein the priming module comprises:
(c) a first nucleic acid strand containing, in a 5′ to 3′ direction, Domain 2B, Domain 1B, Domain 5A, a linker molecule, and Domain 6A, wherein Domain 2B and Domain 1B are respectively complementary to Domain 2A and Domain 1A of the first strand of (a), and Domain 2B is unbound, and
(d) a second nucleic acid containing, in a 5′ to 3′ direction, Domain 6B, Domain 7, Domain 5B and Domain 1A, wherein Domain 6B, Domain 5B and Domain 1A are respectively complementary to and bound to Domain 6A, Domain 5A and Domain 1B of the first strand of (c), and wherein Domain 7 is optionally unbound.

9. The primer of claim 1, wherein the sensing module is linked to the priming module via a linker molecule.

10. The primer of claim 9, wherein the linker molecule is a chemical linker.

11. The primer of claim 9, wherein the linker molecule is a single-stranded nucleic acid.

12. (canceled)

13. (canceled)

14. A method comprising combining in a reaction mixture a target nucleic acid and the primer of claim 1.

15. A method comprising combining in a reaction mixture a target nucleic acid and a primer that comprises a sensing module and a priming module, wherein the sensing module binds to a target nucleic acid and, when bound to the target nucleic acid, recruits and activates the priming module, wherein the activated priming module binds to the target nucleic acid upstream of the sensing module.

16. A method, comprising combining in a reaction mixture a target nucleic acid with a primer that comprises a sensing module and a priming module, wherein
the sensing module comprises a partially double-stranded nucleic acid comprising a first nucleic acid strand bound to a second nucleic acid strand, wherein the first strand comprises
(a) a 5′ domain that includes sequence complementary to the priming module, wherein a portion of the sequence of (a) is complementary to and bound to the second strand, and
(b) a 3′ domain that includes sequence complementary to the target nucleic acid, wherein a portion of the sequence of (b) is complementary to and bound to the second strand, and
the priming module comprises a partially double-stranded nucleic acid comprising a first nucleic acid strand bound to a second nucleic acid strand, wherein the first strand comprises
(c) a 5′ domain that includes sequence complementary to the sensing module,
wherein a portion of the sequence of (c) is complementary to and bound to the second strand, and
(d) a 3′ domain that includes a chemical linker attached to a minimal primer sequence, wherein the minimal primer sequence is complementary to the target nucleic acid and is bound to the second strand.

17. The method of claim 16 further comprising incubating the reaction mixture under conditions that result in recruitment of the primer module to the sensing module, activation of the priming module, and binding of the minimal primer sequence to the target nucleic acid.

18. The method of claim 17 further comprising incubating the reaction mixture under conditions that result in amplification of the target nucleic acid.

19. A composition comprising the primer of claim 1.

20. The composition of claim 19 further comprising the target nucleic acid.

21. A kit comprising the primer of claim 1.

22. A kit comprising at least two of the primer of claim 1, wherein each primer is designed to bind to a different target nucleic acid.

23. The kit of claim 21 further comprising at least one of the following reagents: buffer, deoxyribonucleotide triphosphates (dNTPs), nuclease-free water and polymerase.

24. The method of claim 18, wherein the conditions that result in amplification of the target nucleic acid include incubating the reaction mixture at a temperature of 50° C.-70° C. for a time sufficient to results in amplification of the target nucleic acid.


SEQUENCE LISTING (25 sequences)

SEQ ID NO: 1 - DNA, 52 bp, Artificial Sequence
  Synthetic Polynucleotide
  misc_feature (52)..(52): n is an inverted dT
gcaatcgtcgccctactatcctcctcgttcaaactgatgggacccactccan

SEQ ID NO: 2 - DNA, 27 bp, Artificial Sequence
  Synthetic Polynucleotide
  misc_feature (27)..(27): n is an inverted dT
atcagtttgaacgaggaggatagtagn

SEQ ID NO: 3 - DNA, 48 bp, Artificial Sequence
  Synthetic Polynucleotide
  misc_feature (35)..(36): modified by /iSp18/ /iSp18/ linker
gaggaggatagtagggcgacgattgcatctagtccctcagagttgcag

SEQ ID NO: 4 - DNA, 29 bp, Artificial Sequence
  Synthetic Polynucleotide
  misc_feature (29)..(29): n is an inverted dT
ctgcaactctgagtagatgcaatcgtcgn

SEQ ID NO: 5 - DNA, 65 bp, Artificial Sequence
  Synthetic Polynucleotide
  misc_feature (65)..(65): n is an inverted dT
tgatccgatgacagggcaaatacgagatacttactacacctcagatatatttcttcatgaagacn

SEQ ID NO: 6 - DNA, 27 bp, Artificial Sequence
  Synthetic Polynucleotide
  misc_feature (27)..(27): n is an inverted dT
aggtgtagtaagtatctcgtatttgcn

SEQ ID NO: 7 - DNA, 47 bp, Artificial Sequence
  Synthetic Polynucleotide
  misc_feature (34)..(35): modified by /iSp18/ /iSp18/ linker
tctcgtatttgccctgtcatcggatcaagctagttccatggtgcaag

SEQ ID NO: 8 - DNA, 32 bp, Artificial Sequence
  Synthetic Polynucleotide
  misc_feature (32)..(32): n is an inverted dT
cttgcaccatggttagcttgatccgatgacan

SEQ ID NO: 9 - DNA, 67 bp, Artificial Sequence
  Synthetic Polynucleotide
  misc_feature (67)..(67): n is an inverted dT
ggactagatatccatgcaatcgtcgccctactatcctcctcgttcaaactgatgggacccactccan

SEQ ID NO: 10 - DNA, 38 bp, Artificial Sequence
  Synthetic Polynucleotide
  misc_feature (25)..(26): modified by /iSp18/ /iSp18/ linker
cgacgattgcatggatatctagtccctcagagttgcag

SEQ ID NO: 11 - DNA, 27 bp, Artificial Sequence
  Synthetic Polynucleotide
  misc_feature (27)..(27): n is an inverted dT
atcagtttgaacgaggaggatagtagn

SEQ ID NO: 12 - DNA, 76 bp, Artificial Sequence
  Synthetic Polynucleotide
  misc_feature (76)..(76): n is an inverted dT
actagctgacagttgatccgatgacagggcaaatacgagatacttactacacctcagatatatttcttcatgaagn

SEQ ID NO: 13 - DNA, 38 bp, Artificial Sequence
  Synthetic Polynucleotide
  misc_feature (25)..(26): modified by /iSp18/ /iSp18/ linker
gtcatcggatcaactgtcagctagttccatggtgcaag

SEQ ID NO: 14 - DNA, 26 bp, Artificial Sequence
  Synthetic Polynucleotide
  misc_feature (26)..(26): n is an inverted dT
ggtgtagtaagtatctcgtatttgcn

SEQ ID NO: 15 - DNA, 190 bp, Artificial Sequence
  Synthetic Polynucleotide
ctcagagttgcagatatccggtcgcctaagccagacaactgttcaaactgatgggacccactccatcgagatttctctgtagctagaccaaaatcacctatttttactgtgaggtcttcatgaagaaatatatctgaggtgtagtaagtaaaggaaaacagacggctcgactgatatcttgcaccatgga

SEQ ID NO: 16 - DNA, 171 bp, Artificial Sequence
  Synthetic Polynucleotide
ctcagagttgcagatatccggtcgcctaagtgacaaagaacagctcaaagcaatttctacacgagatcctctctctgaaatcactgagcaggagaaagattttctatggagtcacaggtaagtgctaaaatggagattctcgacggctcgactgatatcttgcaccatgga

SEQ ID NO: 17 - DNA, 37 bp, Artificial Sequence
  Synthetic Polynucleotide
  misc_feature (25)..(26): modified by /iSp18/ /iSp18/ linker
gtcatcggatcaactgtcagctagttccatggtgcaa

SEQ ID NO: 18 - DNA, 190 bp, Artificial Sequence
  Synthetic Polynucleotide
ctcagagttgcagatatccggtcgcctaagccagacaactgttcaaactgatgggacccactccatcgagatttcactgtagctagaccaaaatcacctatttttactgtgaggtcttcatgaagaaatatatctgaggtgtagtaagtaaaggaaaacagacggctcgactgatatcttgcaccatgga

SEQ ID NO: 19 - DNA, 59 bp, Artificial Sequence
  Synthetic Polynucleotide
  misc_feature (57)..(57): modified by 2′-OMe
  misc_feature (58)..(58): modified by 2′-OMe
  misc_feature (59)..(59): n is an inverted dT
gggacccactccatcgagatttcactgtagctagaccaaaatccaagcgacgagaaaan

SEQ ID NO: 20 - DNA, 44 bp, Artificial Sequence
  Synthetic Polynucleotide
  misc_feature (42)..(42): modified by 2′-OMe
  misc_feature (43)..(43): modified by 2′-OMe
  misc_feature (44)..(44): n is an inverted dT
ctcgtcgcttggattttggtctagctacagtgaaatctcgaaan

SEQ ID NO: 21 - DNA, 64 bp, Artificial Sequence
  Synthetic Polynucleotide
ttcatcagtgatcaccgcccatccgacgctatttgtgccgatatctaagcctattgagtatttc

SEQ ID NO: 22 - DNA, 64 bp, Artificial Sequence
  Synthetic Polynucleotide
ttcatcagtgatcaccgcccatccgacgctatttgtgccgctatctaagcctattgagtatttc

SEQ ID NO: 23 - DNA, 30 bp, Artificial Sequence
  Synthetic Polynucleotide
gcttagatatcggcacaaatagcgtcggat

SEQ ID NO: 24 - DNA, 44 bp, Artificial Sequence
  Synthetic Polynucleotide
gcttagatatcggcacaaatagcgtcggatgggcgtcttcttca

SEQ ID NO: 25 - DNA, 32 bp, Artificial Sequence
  Synthetic Polynucleotide
tgaagaagacgcccatccgacgctatttgtgc